(19) United States
(12) Patent Application Publication
(10) Pub. No.: US 2022/0287298 A1
(43) Pub. Date: Sep. 15, 2022

(54) TISSUE CONTAINER SYSTEMS

(71) Applicant: Stratatech Corporation, Madison, WI (US)

(72) Inventors: Justin Koepsel, Madison, WI (US); Kenneth Gratz, Madison, WI (US)

(21) Appl. No.: 17/827,270

(22) Filed: May 27, 2022

Related U.S. Application Data
(63) Continuation-in-part of application No. 16/481,405, filed on Jul. 26, 2019.
(60) Provisional application No. 62/451,379, filed on Jan. 27, 2017.
(60) Provisional application No. 63/194,682, filed on May 28, 2021.

(51) Int. Cl. A01N 1/02; B65D 77/04

(52) U.S. Cl. CPC A01N 1/0273; B65D 77/0406

(57) ABSTRACT

The present disclosure relates generally to tissue container systems and kits that find use in the transport of tissues and methods of using the tissue container systems. In particular, systems and kits that support the transport, thawing and use of cryopreserved human skin equivalents, and methods of their use by a health care provider are provided.

### CROSS REFERENCE TO RELATED APPLICATIONS

This application is a continuation-in-part of U.S. patent application Ser. No. 16/481,405, filed Jul. 26, 2019, which claims priority to PCT/US2018/015490, filed Jan. 26, 2018, which claims the benefit of U.S. provisional application No. 62/451,379, filed Jan. 27, 2017. This application also claims the benefit of U.S. provisional application No. 63/194,682, filed May 28, 2021. The contents of each or which are hereby incorporated by reference in their entirety.

### FIELD OF THE DISCLOSURE

The present invention relates generally to tissue container systems that find use in the transport of tissues and methods of using the tissue container systems. In particular the present invention relates to systems that support the transport, thawing and use of cryopreserved human skin equivalents, and methods of their use by a health care provider.

### BACKGROUND

A major impediment to the acceptance of engineered tissues by medical practitioners, healthcare providers, and second party payers is the lack of a means to effectively and efficiently preserve and store engineered tissues. The nature of living cells and tissue products makes development of long-term storage challenging. Current engineered tissues must often be stored and shipped under carefully controlled conditions to maintain viability and function. Typically, engineered tissue products take weeks or months to produce but must be used within hours or days after manufacture. As a result, tissue engineering companies must continually operate with their production facilities at top capacity and absorb the costs of unsold product which must be discarded. As one specific example, APLIGRAF requires about four weeks to manufacture, is usable for only 15 days and must be maintained between 20 and 23° C. until used. As another example, EPICEL is transported by a nurse from Genzyme Biosurgery's production facility in Cambridge, Mass. to the point of use in a portable incubator and is used immediately upon arrival. Such constraints represent significant challenges to developing convenient and cost-effective products.

Cryopreservation has been explored as a solution to the storage problem, but it is known to induce tissue damage through ice formation, chilling injury, and osmotic imbalance. Besides APLIGRAF, the only other approved full-thickness living skin equivalent, ORCEL, has been evaluated as a frozen product but had the drawback that it must be maintained at temperatures below −100° C. prior to use. This requires specialized product delivery and storage conditions, including use of liquid nitrogen for storage, which is expensive and not readily available in rural clinics and field hospitals.

Accordingly, what is needed in the art are improved methods of cryopreserving viable engineered tissues and cells for storage under conditions that are routinely available at the point of use.

### SUMMARY OF THE DISCLOSURE

The present invention relates generally to tissue container systems that find use in the transport of tissues and their subsequent use by a health care provider, and in particular to systems that support the transport, thawing and use of cryopreserved human skin equivalents.

Accordingly, in some embodiments, the present invention provides tissue containers comprising: a perimeter wall and a substantially planar bottom surface defining a dish, the perimeter wall having a male end and a female end, the male end of the perimeter wall having projecting therefrom a ridge having a length and width, wherein the female end of the perimeter wall defines a space corresponding to the length and width of the ridge so that when an identical tissue container is placed on top of the tissue container the female end of the tissue container releasably receives the ridge extending from the male end of the identical tissue container, and the bottom surface having a perimeter and comprising a perimeter ledge extending around the perimeter to provide a reservoir defined by the perimeter ledge and the bottom surface. In some embodiments, the perimeter wall has a flange extending therefrom. In some embodiments, the flange comprises one or more tabs extending from the male end of the perimeter wall. In some embodiments, the flange comprises one or more tabs extending from the female end of the perimeter wall. In some embodiments, the ridge has a proximal end and the proximal end of the ridge has one or more indents therein.

In some embodiments, the present invention provides tissue container assemblies comprising: substantially identical top and bottom tissue containers, each of the top and bottom tissue containers comprising a perimeter wall and a substantially planar bottom surface defining a dish, the bottom surface having a perimeter and comprising a perimeter ledge extending around the perimeter to provide a reservoir defined by the perimeter ledge and the bottom surface, and the perimeter wall having a male end and a female end, the male end of the perimeter wall having projecting therefrom a ridge having a length and width, wherein the female end of the perimeter wall defines a space corresponding to the length and width of the ridge so that when the top tissue container is placed on the bottom tissue container the female end of the bottom tissue container releasably receives the ridge extending from the male end of the top tissue container. In some embodiments, the perimeter wall of the top tissue container has a top flange extending therefrom and the perimeter wall of the bottom tissue container has a bottom flange extending therefrom so that when the top and bottom tissue containers are assembled the top and bottom flanges contact one another. In some embodiments, the top flange comprises one or more tabs extending from the male end of the perimeter wall and one or more tabs extending from the female end of the perimeter wall. In some embodiments, the bottom flange comprises one or more tabs extending from the male end of the perimeter wall and one or more tabs extending from the female end of the perimeter wall. In some embodiments, the bottom flange comprises one or more tabs extending from the male end of the perimeter wall and one or more tabs extending from the female end of the perimeter wall and the wherein the top flange comprises one or more tabs extending from the male end of the perimeter wall and one or more tabs extending from the female end of the perimeter wall so that when the top and bottom tissue containers are assembled the tabs are offset.

In some embodiments, the present invention provides tissue container systems comprising: substantially identical top and bottom tissue containers and a tray comprising a porous bottom surface, each of the top and bottom tissue containers comprising a perimeter wall and a substantially planar reservoir bottom surface defining a dish, the reservoir bottom surface having a perimeter and comprising a perimeter ledge extending around the perimeter to provide a reservoir defined by the perimeter ledge and the reservoir bottom surface, wherein the tray is sized to be supported by the ledge and above the reservoir bottom surface when inserted into the tissue container, and the perimeter wall having a male end and a female end, the male end of the perimeter wall having projecting therefrom a ridge having a length and width, wherein the female end of the perimeter wall defines a space corresponding to the length and width of the ridge so that when the top tissue container is placed on the bottom tissue container the female end of the bottom tissue container releasably receives the ridge extending from the male end of the top tissue container. In some embodiments, the perimeter wall of the top tissue container has a top flange extending therefrom and the perimeter wall of the bottom tissue container has a bottom flange extending therefrom so that when the top and bottom tissue containers are assembled the top and bottom flanges contact one another. In some embodiments, the top flange comprises one or more tabs extending from the male end of the perimeter wall and one or more tabs extending from the female end of the perimeter wall. In some embodiments, the bottom flange comprises one or more tabs extending from the male end of the perimeter wall and one or more tabs extending from the female end of the perimeter wall. In some embodiments, the bottom flange comprises one or more tabs extending from the male end of the perimeter wall and one or more tabs extending from the female end of the perimeter wall and the wherein the top flange comprises one or more tabs extending from the male end of the perimeter wall and one or more tabs extending from the female end of the perimeter wall so that when the top and bottom tissue containers are assembled the tabs are offset. In some embodiments, the porous bottom surface of the tray is a porous membrane. In some embodiments, the ridge has a proximal end and the proximal end of the ridge has one or more indents therein and the tray has one or more tray tabs so that when the tray is inserted into the bottom tissue container the one or more tabs are inserted into the one or more indents. In some embodiments, the systems further comprise a tissue supported on the porous bottom surface of the tray. In some embodiments, the tissue is cryopreserved. In some embodiments, the tissue is an organotypic skin substitute. The organotypic skin substitute may be a viable, bioengineered, allogenic cellularized scaffold product derived from keratinocytes grown on gelled collagen containing dermal fibroblasts. In some embodiments, the systems further comprise a sterile package containing the tissue container system. The tissue container system can be provided as a kit with one or more absorbent medium and/or one or more liquid media, such as a tissue compatible solution.

In some embodiments, the present invention provides methods of providing a tissue for use by a health care provider comprising packaging a tissue in the tissue container system of the preceding paragraph and providing the packaged tissue to a health care provider in need thereof. In some embodiments, the present invention provides methods of thawing a cryopreserved tissue comprising: providing a cryopreserved tissue in the tissue container system as described above, removing the top tissue container to expose the cryopreserved tissue, optionally transferring the cryopreserved tissue to a new container system, and filling the reservoir in the bottom tissue container with a liquid medium under conditions that the cryopreserved tissue thaws to provide a thawed tissue. In some embodiments, the cryopreserved tissue is an organotypic human skin substitute. In some embodiments, the methods further comprise applying or grafting the organotypic human skin substitute to a burn or a wound on a patient in need thereof.

In some embodiments, the present invention provides a tissue container 100 shown in FIG. 1 that comprises a perimeter wall 105 and a substantially planar bottom surface 110 defining a dish. The perimeter wall 105 has a male end 115 and a female end 120. The male end 115 of the perimeter wall 105 has a ridge 125 extending therefrom that has a length and a width. The female end 120 of the perimeter wall 105 defines a space 130 corresponding to the length and width of the ridge 125 so that when an identical tissue container is placed on top of the tissue container 100 the space 130 provided in said female end 120 of the tissue container can releasably receive the ridge 125 extending from the male end of the identical tissue container as described in more detail below. The bottom surface 110 comprises a perimeter ledge 135 extending around the perimeter of the bottom surface 110. The perimeter ledge 135 forms a reservoir 140 on the bottom of the container that is preferably about 0.50 to 1.5 mm deep, and most preferably about 0.75 mm deep and which can be filled with a liquid medium. The perimeter wall 105 preferably has a flange 145 extending therefrom. In some embodiments, the tissue container 100 further comprises (a) a flange 145 comprising one or more tabs 150 extending the male end 115 and female end 120 of the perimeter wall, (b) a ridge 125 that has one more indents 155 therein that are configured to receive tabs on a tray, (c) a perimeter wall 105 comprising a plurality of grip projections 160, preferably positioned on the male end 115 of the perimeter wall 105, or (d) any combination thereof. The present invention also provides a tissue container assembly comprising substantially identical bottom and top containers, wherein the bottom and top containers are a tissue container described in this paragraph. The present invention also provides a tissue container system shown in FIG. 4 comprising a tissue container assembly of this paragraph and a tray 410. The tray is sized so that it rests on top of the perimeter ledge on the bottom surface of the bottom container as described above. The tray 410 comprises sidewalls 415. Tabs 420 extend from the sidewalls 415 so that they engage and are inserted into indents 425 in the ridge 430 on the male end 435 of the bottom container 405. The tray has a porous bottom surface 440, which is optionally a porous membrane. An identical top container can be placed on the bottom container and closed, without interference from the contained tray. The tissue container system can be optionally sealed, preferably heat sealed, in a sterile bag to provide a primary package. The primary package can be optionally sealed inside a secondary bag. The tissue container system or package containing the tissue container system can be provided as a kit with one or more absorbent medium and/or one or more liquid media, such as a tissue compatible solution.

In some embodiments, the present invention provides methods of providing a tissue for use by a health care provider comprising packaging a tissue in the tissue container system as described in the preceding paragraph and providing the packaged tissue to a health care provider in need thereof. In some embodiments, the present invention provides methods of providing a tissue for use to treat a wound or a burn comprising packaging a tissue in the tissue container system as described in the preceding paragraph and providing the packaged tissue to a health care provider for use to treat wound or a burn. In some embodiments, the present invention provides a method of thawing a cryopreserved skin equivalent prior to application to a subject. The method comprises providing a cryopreserved tissue, preferably an organotypically cultured skin equivalent, in a tissue container system as described in the preceding paragraph, removing the top tissue container to expose the cryopreserved tissue, and filling the reservoir in the bottom tissue container with a liquid medium under conditions that the cryopreserved tissue thaws to provide a thawed tissue, where the cryoprotectant contained within the tissue is diluted into the liquid medium, leaving a tissue that is substantially free of cryoprotectant. In other embodiments, the method comprises removing a primary or secondary package containing a tissue container system comprising a cryopreserved tissue from a freezer or shipping container, removing the tissue container system from the package(s), removing the top tissue container to expose the cryopreserved tissue, and transferring the tray with the cryopreserved skin equivalent from the first tissue container into a second tissue container that is sterile and staged in the sterile field and contains a liquid medium in the container reservoir, such that the transferred cryopreserved tissue thaws to provide a thawed tissue and the cryoprotectant contained within the tissue is diluted into the liquid medium. In some of the above embodiments, the liquid medium is a tissue compatible solution, preferably a buffered solution. In still other embodiments, the tray with the cryopreserved skin equivalent is removed from the tissue container and placed on an absorbent medium to remove thawed cryoprotectant solution from the skin equivalent. The absorbent medium may be in any suitable, preferably sterile, vessel (e.g., a culture vessel or a fresh tissue container assembly). The present invention is not limited to the use of a particular absorbent medium. The absorbent medium preferably comprises a tissue-compatible solution.

Further provided herein is a kit that may include a product dish operable to support an insert tray. The insert tray may contain a polycarbonate membrane and an allogeneic cellularized scaffold product loosely adhered to the polycarbonate membrane. In an embodiment, the insert tray may further comprise polystyrene. The foil pouch may be further packaged in an outer carton. The polycarbonate membrane may form the bottom surface of the insert tray. The product dish may comprise a reservoir formed by a perimeter ledge on a bottom surface of the product dish. The insert tray may rest on the peripheral ledge of the product dish. The product dish (with the insert tray and allogenic cellularized scaffold product) may be packaged in a foil pouch. The allogeneic cellularized scaffold product comprises NIKS cells and/or dermal fibroblasts.

In some embodiments, the kit further comprises a hold solution and a hold dish. The hold solution may be packaged in a bottle contained within a laminated, foil pouch. The hold dish may comprise identical top and bottom portions. The hold dish may be packaged in a clear pouch. The hold dish comprises a reservoir formed by a peripheral ledge on a bottom surface of the hold dish. The reservoir is operable to contain the hold solution. The insert tray may configured to rest on the peripheral ledge of the hold dish.

Other aspects and iterations of the invention are described more thoroughly below.

### BRIEF DESCRIPTION OF THE FIGURES

FIG. 1 is a perspective view of a tissue container in accordance with one embodiment.

FIG. 2 is disassembled perspective view of a tissue container assembly according to one embodiment.

FIG. 3 is a perspective view of an assembled tissue container assembly according to one embodiment.

FIG. 4 is a perspective view of a tissue container with an inserted tray according to one embodiment.

FIG. 5 is a graph of tissue viability after 1-day re-culture. Data are mean±stdev of 15 samples per group (5 samples/tissue×3 tissues/condition in each batch).

FIG. 6 is a graph of post-thaw VEGF secretion during 1-day re-culture. Data are mean±stdev of 3 tissues per condition in each batch.

FIG. 7A and FIG. 7B are graphs of post-thaw tissue barrier function after 1-day re-culture with initial DPM (FIG. 7A) and DPM change (FIG. 7B). Data are mean±stdev of 12 reads per group (4 samples/tissue×3 tissues/condition in each batch).

FIG. 8A shows packaging for a product dish, including a foil pouch and outer carton according to one embodiment.

FIG. 8B shows a product dish within the foil pouch according to one embodiment.

FIG. 8C shows an insert tray and product dish according to one embodiment.

FIG. 8D shows an insert tray according to one embodiment.

FIG. 9A shows a hold solution bottle in a foil pouch according to one embodiment.

FIG. 9B shows a hold dish packaged in a clear pouch according to one embodiment.

FIG. 9C shows the hold solution being poured into the hold dish according to one embodiment.

FIG. 9D shows the insert tray being placed in the hold dish according to one embodiment.

### DETAILED DESCRIPTION

It is to be understood that this disclosure is not limited to the particular methods, compositions, or materials specified herein, but is extended to equivalents thereof as would be recognized by those ordinarily skilled in the relevant arts. It should also be understood that terminology employed herein is used for the purpose of describing particular embodiments only and is not intended to be limiting.

The present invention relates generally to tissue container systems and kits that find use in the transport of tissues and their subsequent use by a health care provider, and in particular to systems that support the transport, thawing and use of cryopreserved human skin equivalents.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/m L” The endpoint may also be based on the variability allowed by an appropriate regulatory body, such as the FDA, USP, etc.

As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, the terms “skin equivalent,” “human skin equivalent,” “human skin substitute,” “organotypic human skin equivalent,” “organotypic skin substitute,” “allogeneic cultured keratinocyte composition,” “allogeneic cellularized scaffold product,” and StrataGraft® skin tissue are used interchangeably to refer to an in vitro derived culture of keratinocytes that has stratified into squamous epithelia. Typically, the skin equivalents are produced by organotypic culture and include a dermal layer in addition to a keratinocyte layer.

As used herein, the term “sterile” refers to a skin equivalent that is essentially or completely free of detectable microbial or fungal contamination.

As used herein, the term “NIKS cells” refers to cells having the characteristics of the cells deposited as cell line ATCC CRL-12191. “NIKS” stands for near-diploid immortalized keratinocytes and is a registered trademark.

As used herein, the term “viable” when used in reference to a skin equivalent refers to the viability of cells in the skin equivalent following cryopreservation. In preferred embodiments, a “viable” skin has an A550 of at least 50%, 60%, 70%, 80% or 90% of a control non-cryopreserved tissue as measured by an MTT assay or at least 50%, 70%, 80% or 90% of the readout value of a similar viability assay.

As used herein, the term “culture vessel” refers to any vessel of the type commonly used to culture cells or tissues and includes circular, rectangular, and square dishes formed from a suitable material such as tissue culture plastic, polystyrene, polymers, plastics, glass, etc. The term “culture vessel” and “growth chamber” are used interchangeably. Tissue containers of the present disclosure are not culture vessels, as used herein, at least because the tissue containers of the present disclosure are not of a suitable size for long-term culture.

As used herein, the terms “tissue container,” “tissue container assembly,” “product dish,” and “hold dish” are used interchangeably to refer to a container or multiple containers used to store, transport, and/or thaw a tissue (e.g. an allogeneic cellularized scaffold product). Multiple tissue containers may be used in the processes of storing and thawing the tissue. For example, an allogeneic cellularized scaffold product may be cryopreserved and stored in a first tissue container and a second tissue container may be configured to connect to the first tissue container to act as a lid to the first tissue container, such that a tissue container assembly includes the first and second containers acting as identical top and bottom portions. In addition, a hold dish may be a tissue container without an allogeneic cellularized scaffold product stored within it. The hold dish may include two tissue containers acting as identical top and bottom portions.

As used herein, the terms “tray”, “insert tray”, and “tissue insert” are used interchangeably to refer to a tray or dish operable to hold the allogeneic cellularized scaffold product and be inserted into, fit within, or rest inside a tissue container.

As used herein, the terms “tissue compatible solution,” “media,” and “hold solution” are used interchangeably to refer to a solution used to thaw the cryopreserved tissue and/or remove cryoprotectant from the tissue.

The tissue containers of the instant disclosure make efficient use of freezer and surgical suite space as they are approximately 60% smaller than previously utilized containers. The tissue containers are compatible with a tray that includes a porous membrane as bottom surface upon which a tissue (e.g., an organotypic skin substitute/allogeneic cellularized scaffold product) can be supported. The other surfaces of the tray are preferably clear or translucent plastics produced by a thermoforming process from a plastic sheet, injection molding, or other methods known in the art to manipulate plastics. Suitable plastics include medical grade plastics, for example, polyethylene terephthalate glycol-modified (PETG), polystyrene, etc. In some preferred embodiments, the tray is a preferably an insert tray as described herein below. The tissue containers include a reservoir that can be filled with media to thaw the tissue in the container and remove cryoprotectant when the tissue has been frozen. This provides an advantage over previous systems used for thawing tissues where the tissue had to be removed from the container in the surgical sterile field and then placed on a Telfa® pad. The tissue containers of the present invention preferably include a top and bottom which are mirror images of one another. The top and bottom pieces of the container assembly are substantially identical and can be snapped together to form an enclosed container. The use of a top and bottom which are substantially identical means that both the top and bottom piece can be produced from the same molds, which creates efficiencies during the production of the top and bottom pieces. The top and bottom pieces are preferably clear and produced by a thermoforming process from a plastic sheet. Suitable plastics for forming the tissue containers include medical grade thermoformable plastics, for example, polyethylene terephthalate glycol-modified (PETG). In one embodiment, the tissue container may be a PETG rectangular tray. The tissue containers and/or the insert tray may be sterilized using gamma irradiation. Accordingly, the present invention provides improved tissue containers, tissue container systems, and kits which will be described in more detail below.

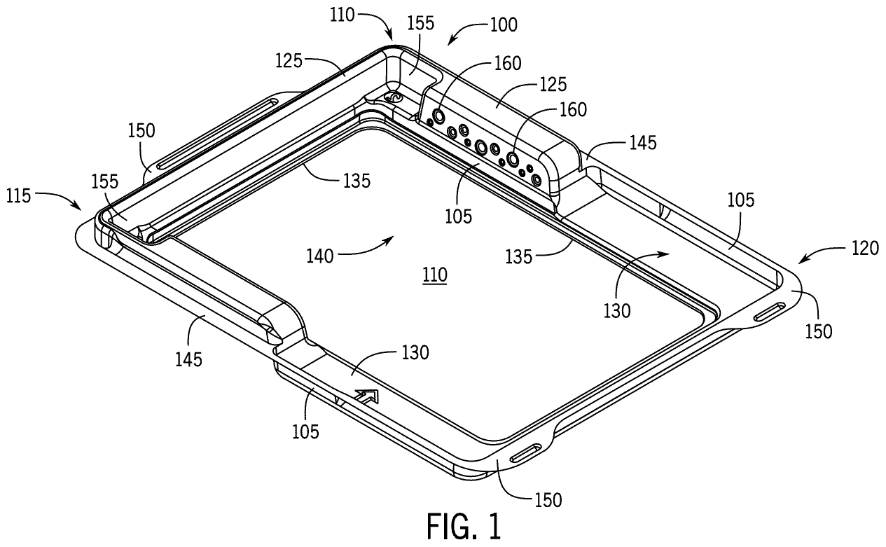

FIG. 1 shows a tissue container 100. In some embodiments, the tissue container 100 preferably comprises a perimeter wall 105 and a substantially planar bottom surface 110 defining a dish. The perimeter wall 105 has a male end 115 and a female end 120. The male end 115 of the perimeter wall 105 has a ridge 125 extending therefrom that has a length and a width. The female end 120 of the perimeter wall 105 defines a space 130 corresponding to the length and width of the ridge 125 so that when an identical tissue container is placed on top of the tissue container 100 the space 130 provided in said female end 120 of the tissue container can releasably receive the ridge 125 extending from the male end of the identical tissue container as described in more detail below. The bottom surface 110 comprises a perimeter ledge 135 extending around the perimeter of the bottom surface 110. The perimeter of the bottom surface 110 is defined by the perimeter wall 105. The perimeter ledge 135 extends in a direction perpendicular to the perimeter wall 105 along the bottom surface 110, such that the perimeter ledge 135 abuts the perimeter wall 105. The perimeter ledge 135 may have a height of about 0.50 to 1.5 mm. In one example, the perimeter ledge 135 has a height of 0.75 mm. Thus, the perimeter ledge 135 forms a reservoir 140 on the bottom of the container that is preferably about 0.50 to 1.5 mm deep, and most preferably about 0.75 mm deep and which can be filled with a liquid medium. The perimeter wall 105 preferably has a flange 145 extending therefrom. In some embodiments, the flange 145 comprises one or more tabs 150 extending the male end 115 and female end 120 of the perimeter wall. In some embodiments, the ridge 125 has one or more indents 155 therein that are configured to receive tabs on a tray, which is described in more detail below. In some further embodiments the perimeter wall 105 preferably comprises a plurality of grip projections 160, preferably positioned on the male end 115 of the perimeter wall 105.

In some embodiments, the tray may be configured to rest on the perimeter ledge 135. As described below, the bottom surface of the tray may be porous. The reservoir 140 may be filled with a hold solution when the container is used to thaw the tissue, and the hold solution may surround and/or be within the tray when the tray rests on the perimeter ledge 135.

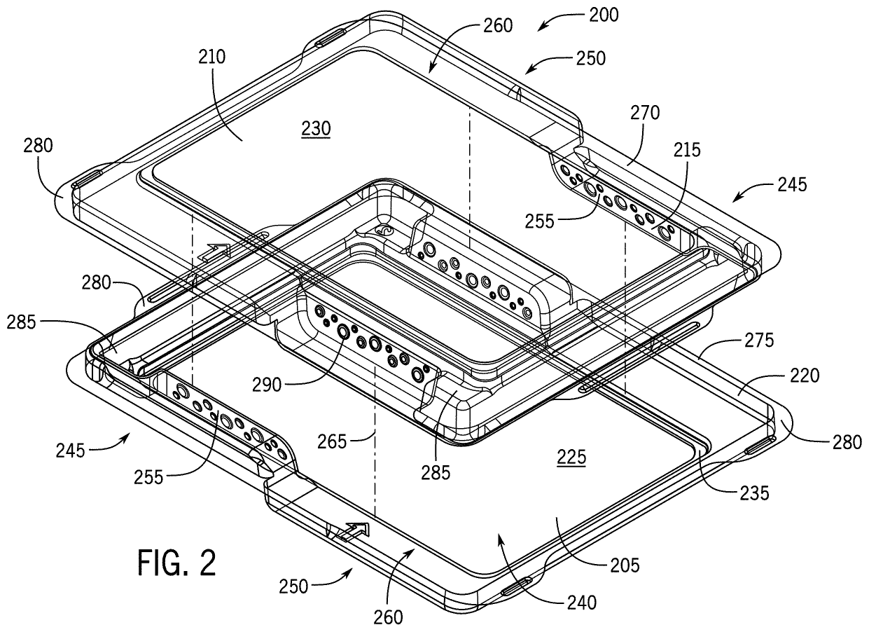

FIG. 2 shows an expanded view of a tissue container assembly 200 of the instant invention. The tissue container assembly 200 preferably comprises substantially identical bottom and top containers 205 and 210. Each of the bottom and top containers 205 and 210 comprise a perimeter wall 215 and 220 and have a bottom surface 225 in the case of the bottom container 205 and a top surface 230 in the case of the top container 210. The bottom surface 225 comprises a perimeter ledge 235 extending around the perimeter of the bottom surface 225. The perimeter of the bottom surface 225 is defined by the perimeter wall 215. The perimeter ledge 235 extends in a direction perpendicular to the perimeter wall 215 along the bottom surface 225, such that the perimeter ledge 235 abuts the perimeter wall 215. The perimeter ledge 235 may have a height of about 0.50 to 1.5 mm. In one example, the perimeter ledge 235 has a height of 0.75 mm. The perimeter ledge 235 forms a reservoir 240 on the bottom of the bottom container 205 that is preferably about 0.50 to 1.5 mm deep, and most preferably about 0.75 mm deep and which can be filled with a liquid medium. Each of the bottom and top containers 205 and 210 comprise male and female ends 245 and 250. The male ends 245 have a ridge 255 extending therefrom that has a length and a width. The female ends 250 define a space 260 corresponding to the length and width of the ridges 255 so that when the top container 210 is placed on the bottom container 205 along the alignment shown by dashed lines 265 the space 260 provided in said female ends 250 of the bottom and top tissue containers 205 and 210 can releasably receive the ridges 255 so that the bottom and top containers 205 and 210 can be releasably snapped together. The perimeter walls 215 and 220 preferably have flanges 270 and 275 extending therefrom. In some embodiments, the flanges comprise one or more tabs 280 extending the male and female ends 245 and 250. In some embodiments, the ridges 255 have one or more indents 285 therein that are configured to receive tabs on a tray, which is described in more detail below. In some further embodiments the perimeter walls preferably comprises a plurality of grip projections 290, preferably positioned on the male ends 245.

In some embodiments, the tray may be configured to rest on the perimeter ledge 235. As described below, the bottom surface of the tray may be porous. The reservoir 240 may be filled with a hold solution when the container is used to thaw the tissue, and the hold solution may surround and/or be within the tray when the tray rests on the perimeter ledge 235.

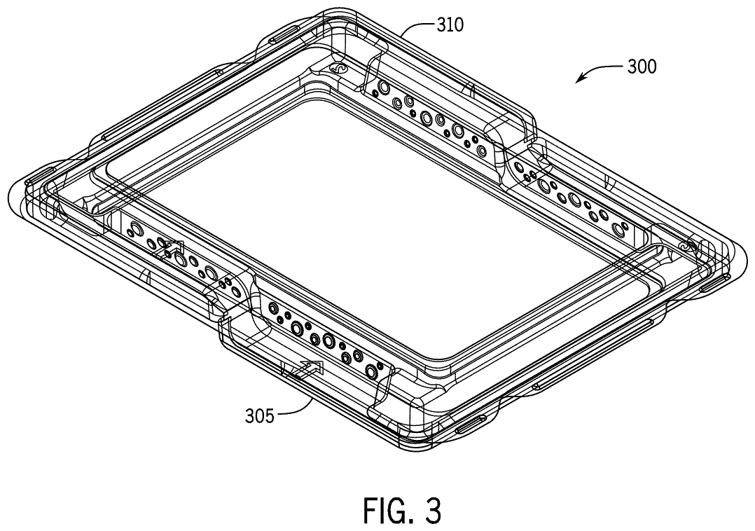

FIG. 3 shows a container assembly 300 of the present invention where the bottom container 305 and top container 310 are fully engaged to form an enclosed container.

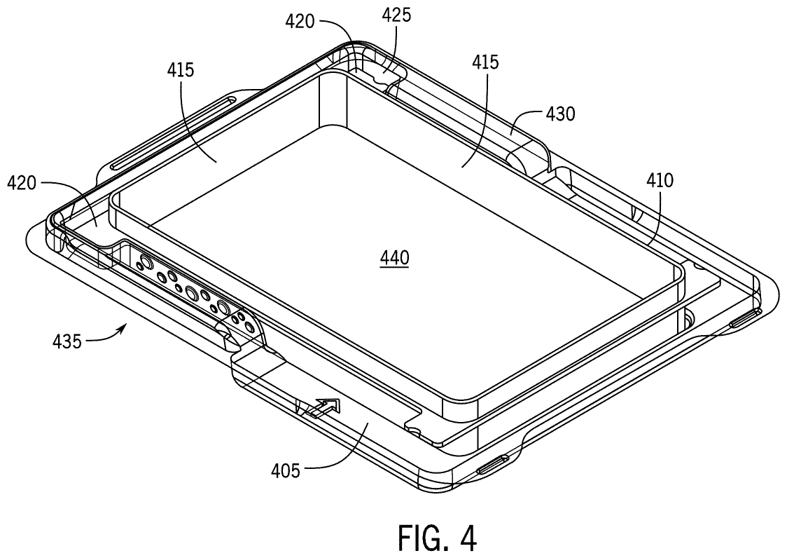

Further provided herein is a tissue container system comprising the bottom and top containers described above along with a tray. FIG. 4 shows a bottom container into which a tray 410 has been inserted. The tray 410 is sized so that it rests on top of the perimeter ledge on the bottom surface of the bottom container as described above. The tray 410 comprises sidewalls 415. Tabs 420 extend from the sidewalls 415 so that they engage and are inserted into indents 425 in the ridge 430 on the male end 435 of the bottom container 405. Sidewalls 415 and tabs 420 are preferably clear or translucent plastics produced by a thermoforming process from a plastic sheet, injection molding, or other methods known in the art to manipulate plastics. Preferred plastics are medical grade thermoformable plastics including, but not limited to, polyethylene terephthalate glycol-modified (PETG) and polystyrene. In some preferred embodiments, the plastic used for sidewalls 415 and tabs 420 is polystyrene.

The tray preferably has a porous bottom surface 440. In some preferred embodiments, the porous bottom surface is a porous membrane, preferably a semi-permeable polymer film, more preferably a semi-permeable track-etched polymer film. The membrane can be tissue culture treated (e.g., plasma treated) to improve cell attachment. In further embodiments, the membrane has a nominal thickness of at least 5 microns, in some example, about 5 microns to about 20 microns, preferably about 10 microns to about 20 microns, more preferably about 10 microns to about 15 microns. In other examples, the membrane has a nominal thickness of about 10 microns. Suitable membrane materials are known in the art and include, but are not limited to, polyethylene terephthalate, polyester, polycarbonate, or any other membrane material used in commercially available, tissue-culture treated inserts (e.g., Transwell®, Snapwell™, etc.) with a multiplicity of open pores therethrough. In at least one example, the membrane is a polycarbonate membrane. Preferably the pores have a nominal pore size of about 0.1 micron to about 10 microns, preferably about 0.1 micron to about 0.8 micron, more preferably about 0.2 micron to about 0.8 micron, even more preferably about 0.4 micron, about 0.5 micron, or about 0.6 micron. In at least one embodiment, the polycarbonate membrane has 0.4 micron pores to allow for media exchange, such as the hold solution. The membrane preferably has a nominal pore density between about 1×10⁸ and about 4×10⁶ pores per square centimeter, though a wider range is also acceptable. Most preferably, a membrane is formed from polycarbonate having pores with a nominal size of about 0.4 micron and a nominal pore density about 1×10⁸ pores per square centimeter. The membrane may be attached to sidewalls 415 by any suitable method known in the art, for example by heat sealing, sonic welding, solvent bonding, adhesive bonding and the like.

The tray is configured to hold a tissue, such as an allogeneic cellularized scaffold product. The allogeneic cellularized scaffold product may be loosely adhered to the membrane on the bottom surface of the tray. The pores within the membrane may allow for a liquid, such as the hold solution, to permeate through the membrane and tray to surround the membrane within. When the hold solution is warmed, it may be used to thaw a cryopreserved allogeneic cellularized scaffold product in the tray. In some embodiments, the tray with the cryopreserved allogeneic cellularized scaffold product may be within a tissue container used to store and transport the tissue. In an embodiment, the tray may be removed from the tissue container to a new tissue container (e.g. hold dish) for thawing. The warmed (e.g. 35° C. to 39° C.) hold solution may be placed in the hold dish and then the tray with the allogeneic cellularized scaffold product may be moved from the first tissue container to the hold dish for thawing.

Further provided herein is a kit for storing, transporting, and/or preparing an allogenic cellularized scaffold product. The tissue containers (e.g. both the product dish and hold dish), tissue, and hold solution may be packaged for storage and transport. In some examples, one or more of these items may be provided together as a kit. The product dish, hold dish, insert tray, and/or foil pouches may be sterilized using gamma irradiation. In some embodiments, the product dish, hold dish, and/or insert tray may not have any detectable extractables or leachables.

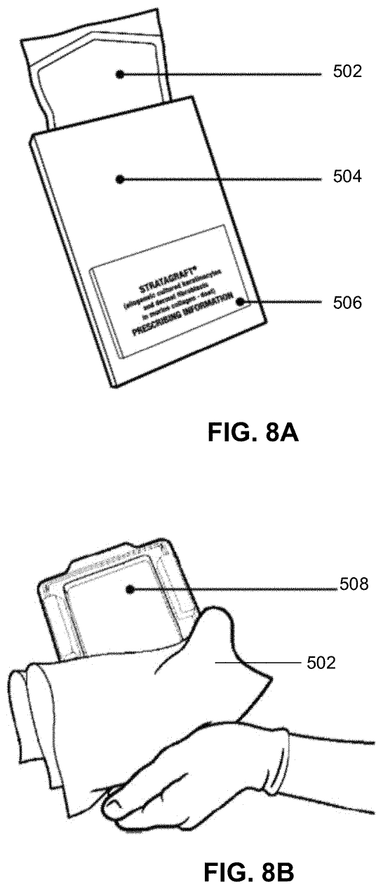

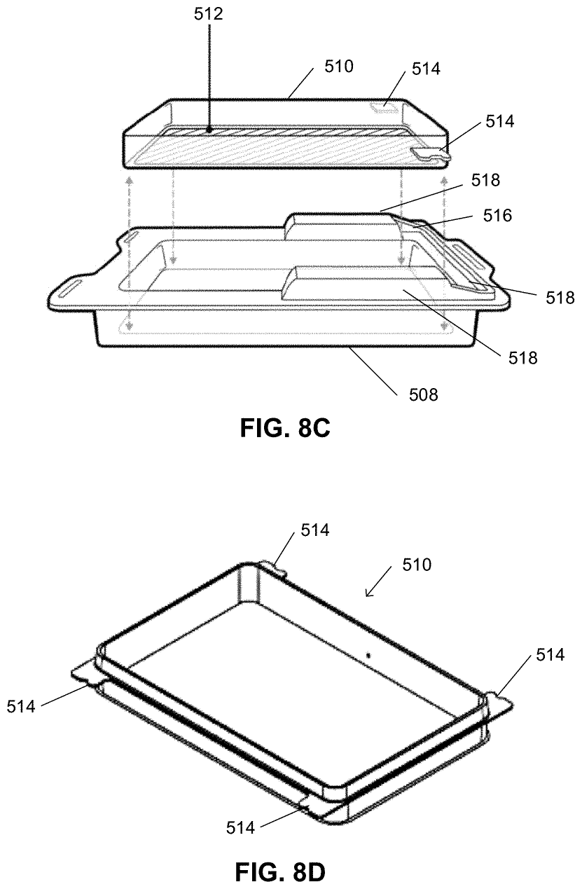

FIG. 8A shows an example of the product dish with an allogenic cellularized scaffold product (not shown) contained in a laminated foil pouch 502, which is then contained in an outer carton 504. The laminated foil pouch may be made of polyethylene, aluminum foil, and/or low density polyethylene. For example, the laminated foil pouch may include a 30 gauge aluminum foil layer. In at least one example, the laminated foil pouch may include 48 ga PET, White LDPE, 0.35 mil aluminum foil, LDPE, and a 2.0 mil sealant layer. The outer carton 504 may be an 18 pt cardboard carton and may contain prescribing information 506 on an outer surface. The outer carton may have an opacity percentage of up to about 99.74%. The allogenic cellularized scaffold product may be cryopreserved and therefore, the product dish, foil pouch 502, and/or outer carton 504 are able to be frozen.

The FIG. 8B shows the product dish 508 within the foil pouch 502. The foil pouch 502 may be peeled open to reveal the product dish 508 when the scaffold product is ready to be used. The product dish 508 may include identical top and bottom portions. In an example, the bottom portion is operable to hold an insert tray 510 with the allogenic cellularized scaffold product 512, as seen in FIG. 8C. The insert tray 510 may contain a polycarbonate membrane (not shown) and the allogeneic cellularized scaffold product 512 product loosely adhered to the polycarbonate membrane. The polycarbonate membrane may form the bottom surface of the insert tray 510. In an embodiment, the insert tray may further comprise polystyrene. The insert tray 510 may be a 100 cm² rectangular insert to hold a 100 cm² scaffold product.

In some embodiments, as seen in FIGS. 8C and 8D, the insert tray 510 has one or more tray tabs 514 so that when the tray 510 is inserted into the bottom of the product dish 508, the one or more tabs 514 are inserted into one or more indents 516 in the ridge(s) 518 on the product dish 508. In at least one example, the insert tray 510 includes two tabs 514 on one end of the tray 510 that fit into two corresponding indents 516 formed in two ridges 518 on one end of the product dish 508. In another example, the insert tray 510 includes four tabs 514, one near each corner of the insert tray 510, where two tabs 514 are operable to fit into two corresponding indents 516 formed in two ridges 518 on one end of the product dish 508 and the other two tabs 514 are operable to rest on the perimeter wall of the product dish 508.

In an embodiment, the product dish 508 may include a reservoir formed by a perimeter ledge on a bottom surface of the product dish 508. In this embodiment, the insert tray 510 may rest on the peripheral ledge of the product dish 508. In other embodiments, the reservoir may be formed by a perimeter wall of the product dish 508 and the insert tray 510 may rest on the bottom surface of the product dish 508. A top portion product dish 508 may be used to cover a bottom portion product dish 508 containing the insert tray 510 and scaffold product 512, such that the scaffold product 512 is fully enclosed in the product dish 508 and further in the foil pouch 502 and outer carton 504.

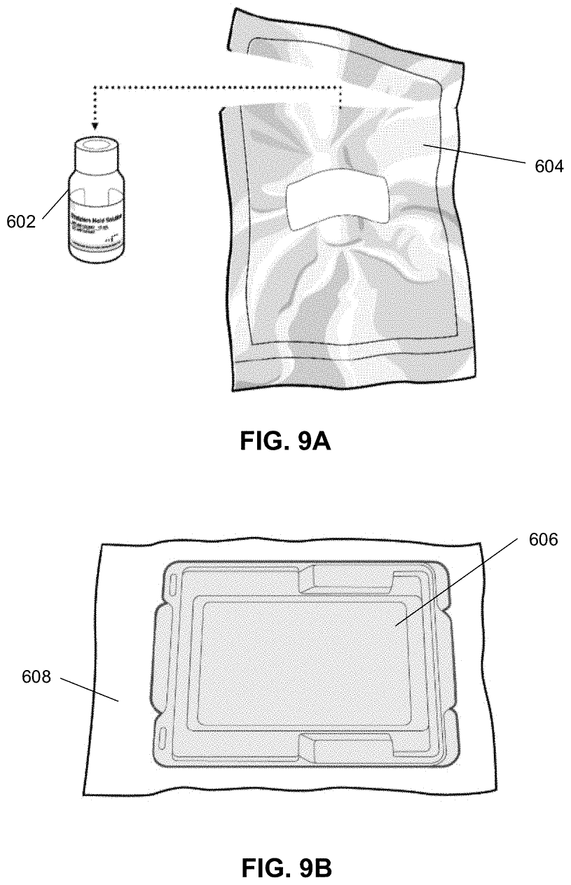

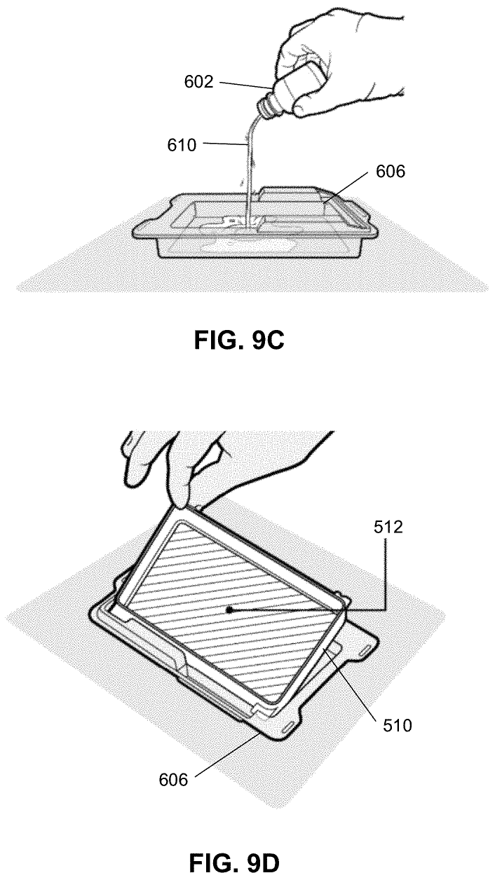

FIG. 9A shows an example hold solution (not shown) provided in a plastic bottle 602 contained within a laminated, foil pouch 604. FIG. 9B shows an example hold dish 606 contained in a clear pouch 608. The hold dish 606 may include identical top and bottom portions. In some examples, the hold dish 606 may be identical to the product dish as shown in FIGS. 8B and 8C. However, the hold dish 606 is not packaged with the insert tray or scaffold product. Because the hold dish 60 is not packaged with the scaffold product, it does not need to be stored at a cold temperature and is able to be packaged in the clear pouch 608 instead of a laminated foil pouch. As seen in FIG. 9C, the hold dish 606 may be operable to receive the hold solution 610 within a reservoir on the bottom surface of the hold dish 606. In an embodiment, reservoir of the hold dish 606 may be formed by a perimeter ledge on a bottom surface of the hold dish 606. In this embodiment, the insert tray 510 may rest on the peripheral ledge of the hold dish 606. In other embodiments, the reservoir may be formed by a perimeter wall of the hold dish 606 and the insert tray 510 may rest on the bottom surface of the hold dish 606. As seen in FIG. 9D, the hold dish 606 may then be operable to receive the insert tray 510 with the scaffold product 512 after it has been removed from the product dish 508. The hold solution 610 within the hold dish 606 may be used to thaw the scaffold product 512 in the insert tray 510 and may be used to remove any remaining cryoprotectant in the scaffold product. The hold solution 610 may be warmed in the plastic bottle 602 prior to being poured into the hold dish 606.

Further provided herein is a kit that may include a product dish operable to support an insert tray with an allogenic cellularized scaffold. The product dish, with the insert tray and allogenic cellularized scaffold product, may be packaged in a foil pouch. The foil pouch may be further packaged in an outer carton. In some embodiments, the kit may further include a hold solution and a hold dish. The hold solution may be packaged in a bottle contained within a laminated, foil pouch. The hold dish may be packaged in a clear pouch. The kit may be provided with one or more of the product dish, insert tray, product scaffold, hold solution, and the hold dish for storage, transport, thawing, and preparation of the cryopreserved product scaffold.

The packaging components which come into contact with the scaffold product are kept in cryopreserved conditions within the pouch and cardboard carton until preparation for clinical use. The allogenic cellularized scaffold product is kept in the foil pouch until preparation for clinical use. The foil pouch may not contact the scaffold product during packaging, shipping, or clinical use. The foil pouch may include a 30 gauge layer of aluminum foil, thus light transmission is not considered a risk for the scaffold product. Additionally, the pouched units may be stored in an 18 pt cardboard carton that has an opacity percentage of 99.74%. The duration of product light exposure is expected to be minimal due to storage of the skin tissue in a −70 to −90° C. freezer until preparation for clinical use. Thus, light transmission is not considered a risk for the scaffold product.

The tissue containers may be used to cryopreserve, store, transport, and/or thaw a variety of tissues. The tissues are preferably supported on the porous bottom surface of the tray and are enclosed with a container assembly comprising bottom and top containers. In some preferred embodiments, the tissues are cryopreserved. In some embodiments, the tissues are skin tissues, for example, cadaver skin or organotypic skin equivalents. In some exemplary embodiments, the tissues are organotypic skin equivalents or cryopreserved organotypic skin equivalents.

The tissue is not limited to any particular organotypic skin equivalent. Indeed, the use of a variety of cell lines and sources that can differentiate into squamous epithelia, including both primary and immortalized keratinocytes are contemplated. In at least one example, the allogenic cellularized scaffold product may be derived from keratinocytes grown on gelled collagen containing dermal fibroblasts. The allogenic cellularized scaffold product may be used for the treatment of adults with thermal burns containing intact dermal elements for with surgical intervention is clinically indicated (deep partial-thickness burns). Sources of cells include keratinocytes and dermal fibroblasts biopsied from humans and cavaderic donors (Auger et al, In Vitro Cell. Dev. Biol.—Animal 36:96-103; U.S. Pat. Nos. 5,968,546 and 5,693,332, each of which is incorporated herein by reference), neonatal foreskins (Asbill et al., Pharm. Research 17(9): 1092-97 (2000); Meana et al., Burns 24:621-30 (1998); U.S. Pat. Nos. 4,485,096; 6,039,760; and 5,536,656, each of which is incorporated herein by reference), and immortalized keratinocytes cell lines such as NM1 cells (Baden, In Vitro Cell. Dev. Biol. 23(3):205-213 (1987)), HaCaT cells (Boucamp et al., J. cell. Boil. 106:761-771 (1988)); and NIKS® cells (Cell line BC-1-Ep/SL; U.S. Pat. No. 5,989,837, incorporated herein by reference; ATCC CRL-12191). Each of the mentioned cell lines can be cultured or genetically modified in order to produce a cell line capable of expressing or co-expressing the desired protein(s). In particularly preferred embodiments, NIKS® cells are utilized. The discovery of the novel NIKS® human keratinocyte cell line provides an opportunity to genetically engineer human keratinocytes with non-viral vectors. A unique advantage of the NIKS® cells is that they are a consistent source of genetically-uniform, pathogen-free human keratinocytes. For this reason, they are useful for the application of genetic engineering and genomic gene expression approaches to provide human skin equivalents with enhanced properties over currently available skin equivalents. NIKS® cells, identified and characterized at the University of Wisconsin, are nontumorigenic, karyotypically stable, and exhibit normal growth and differentiation both in monolayer and organotypic culture. NIKS® cells form fully stratified skin equivalents in culture. These cultures are indistinguishable by all criteria tested thus far from organotypic cultures formed from primary human keratinocytes. Unlike primary cells however, NIKS® cells exhibit an extended lifespan in monolayer culture. This provides an opportunity to genetically manipulate the cells and isolate new clones of cells with new useful properties (Allen-Hoffmann et al., J. Invest. Dermatol., 114(3): 444-455 (2000)).

The NIKS® cells arose from the BC-1-Ep strain of human neonatal foreskin keratinocytes isolated from an apparently normal male infant. In early passages, the BC-1-Ep cells exhibited no morphological or growth characteristics that were atypical for cultured normal human keratinocytes. Cultivated BC-1-Ep cells exhibited stratification as well as features of programmed cell death. To determine replicative lifespan, the BC-1-Ep cells were serially cultivated to senescence in standard keratinocyte growth medium at a density of 3×10⁵ cells per 100-mm dish and passaged at weekly intervals (approximately a 1:25 split). By passage 15, most keratinocytes in the population appeared senescent as judged by the presence of numerous abortive colonies which exhibited large, flat cells. However, at passage 16, keratinocytes exhibiting a small cell size were evident. By passage 17, only the small-sized keratinocytes were present in the culture and no large, senescent keratinocytes were evident. The resulting population of small keratinocytes that survived this putative crisis period appeared morphologically uniform and produced colonies of keratinocytes exhibiting typical keratinocyte characteristics including cell-cell adhesion and apparent squame production. The keratinocytes that survived senescence were serially cultivated at a density of 3×10⁵ cells per 100-mm dish. Typically the cultures reached a cell density of approximately 8×10⁶ cells within 7 days. This stable rate of cell growth was maintained through at least 59 passages, demonstrating that the cells had achieved immortality. The keratinocytes that emerged from the original senescencing population are now termed NIKS®. The NIKS® cell line has been screened for the presence of proviral DNA sequences for HIV-1, HIV-2, EBV, CMV, HTLV-1, HTLV-2, HBV, HCV, B-19 parvovirus, HPV-16, SV40, HHV-6, HHV-7, HPV-18 and HPV-31 using either PCR or Southern analysis. None of these viruses were detected.

Chromosomal analysis was performed on the parental BC-1-Ep cells at passage 3 and NIKS® cells at passages 31 and 54. The parental BC-1-Ep cells have a normal chromosomal complement of 46, XY. At passage 31, all NIKS cells contained 47 chromosomes with an extra isochromosome of the long arm of chromosome 8. No other gross chromosomal abnormalities or marker chromosomes were detected. The karyotype of the NIKS® cells has been shown to be stable to at least passage 54.

The DNA fingerprints for the NIKS® cell line and the BC-1-Ep keratinocytes are identical at all twelve loci analyzed demonstrating that the NIKS® cells arose from the parental BC-1-Ep population. The odds of the NIKS® cell line having the parental BC-1-Ep DNA fingerprint by random chance is 4×10⁻¹⁶. The DNA fingerprints from three different sources of human keratinocytes, ED-1-Ep, SCC4 and SCC13y are different from the BC-1-Ep pattern. This data also shows that keratinocytes isolated from other humans, ED-1-Ep, SCC4, and SCC13y, are unrelated to the BC-1-Ep cells or each other. The NIKS® DNA fingerprint data provides an unequivocal way to identify the NIKS® cell line.

Loss of p53 function is associated with an enhanced proliferative potential and increased frequency of immortality in cultured cells. The sequence of p53 in the NIKS® cells is identical to published p53 sequences (GenBank accession number: M14695). In humans, p53 exists in two predominant polymorphic forms distinguished by the amino acid at codon 72. Both alleles of p53 in the NIKS® cells are wild-type and have the sequence CGC at codon 72, which codes for an arginine. The other common form of p53 has a proline at this position. The entire sequence of p53 in the NIKS® cells is identical to the BC-1-Ep progenitor cells. Rb was also found to be wild-type in NIKS® cells.

Anchorage-independent growth is highly correlated to tumorigenicity in vivo. For this reason, the anchorage-independent growth characteristics of NIKS® cells in agar or methylcellulose-containing medium were investigated. NIKS® cells remained as single cells after 4 weeks in either agar- or methylcellulose-containing medium. The assays were continued for a total of 8 weeks to detect slow growing variants of the NIKS® cells. None were observed.

To determine the tumorigenicity of the parental BC-1-Ep keratinocytes and the immortal NIKS® keratinocyte cell line, cells were injected into the flanks of athymic nude mice. The human squamous cell carcinoma cell line, SCC4, was used as a positive control for tumor production in these animals. The injection of samples was designed such that animals received SCC4 cells in one flank and either the parental BC-1-Ep keratinocytes or the NIKS® cells in the opposite flank. This injection strategy eliminated animal to animal variation in tumor production and confirmed that the mice would support vigorous growth of tumorigenic cells. Neither the parental BC-1-Ep keratinocytes (passage 6) nor the NIKS® keratinocytes (passage 35) produced tumors in athymic nude mice.

NIKS® cells were analyzed for the ability to undergo differentiation in both submerged culture and organotypic culture. Techniques for organotypic culture are described in detail in the examples. In particularly preferred embodiments, the organotypically cultured skin equivalents of the present invention comprise a dermal equivalent formed from collagen or a similar material and fibroblasts. The keratinocytes, for example NIKS® cells or a combination of NIKS® cells and cells from a patient are seeded onto the dermal equivalent and form an epidermal layer characterized by squamous differentiation following the organotypic culture process.

For cells in submerged culture, the formation of cornified envelopes was monitored as a marker of squamous differentiation. In cultured human keratinocytes, early stages of cornified envelope assembly results in the formation of an immature structure composed of involucrin, cystatin-a and other proteins, which represent the innermost third of the mature cornified envelope. Less than 2% of the keratinocytes from the adherent BC-1-Ep cells or the NIKS® cell line produce cornified envelopes. This finding is consistent with previous studies demonstrating that actively growing, subconfluent keratinocytes produce less than 5% cornified envelopes. To determine whether the NIKS® cell line is capable of producing cornified envelopes when induced to differentiate, the cells were removed from adherent culture and suspended for 24 hours in medium made semi-solid with methylcellulose. Many aspects of terminal differentiation, including differential expression of keratins and cornified envelope formation can be triggered in vitro by loss of keratinocyte cell-cell and cell-substratum adhesion. The NIKS® keratinocytes produced as many as and usually more cornified envelopes than the parental keratinocytes. These findings demonstrate that the NIKS® keratinocytes are not defective in their ability to initiate the formation of this cell type-specific differentiation structure.

To confirm that the NIKS® keratinocytes can undergo squamous differentiation, the cells were cultivated in organotypic culture. Keratinocyte cultures grown on plastic substrata and submerged in medium replicate but exhibit limited differentiation. Specifically, human keratinocytes become confluent and undergo limited stratification producing a sheet consisting of 3 or more layers of keratinocytes. By light and electron microscopy there are striking differences between the architecture of the multilayered sheets formed in submerged culture and intact human skin. In contrast, organotypic culturing techniques allow for keratinocyte growth and differentiation under in vivo-like conditions. Specifically, the cells adhere to a physiological substratum consisting of dermal fibroblasts embedded within a fibrillar collagen base. The organotypic culture is maintained at the air-medium interface. In this way, cells in the upper sheets are air-exposed while the proliferating basal cells remain closest to the gradient of nutrients provided by diffusion through the collagen gel. Under these conditions, correct tissue architecture is formed. Several characteristics of a normal differentiating epidermis are evident. In both the parental cells and the NIKS® cell line a single layer of cuboidal basal cells rests at the junction of the epidermis and the dermal equivalent. The rounded morphology and high nuclear to cytoplasmic ratio is indicative of an actively dividing population of keratinocytes. In normal human epidermis, as the basal cells divide they give rise to daughter cells that migrate upwards into the differentiating layers of the tissue. The daughter cells increase in size and become flattened and squamous. Eventually these cells enucleate and form cornified, keratinized structures. This normal differentiation process is evident in the upper layers of both the parental cells and the NIKS® cells. The appearance of flattened squamous cells is evident in the upper epidermal layers and demonstrates that stratification has occurred in the organotypic cultures. In the uppermost part of the organotypic cultures the enucleated squames peel off the top of the culture. To date, no histological differences in differentiation at the light microscope level between the parental keratinocytes and the NIKS® keratinocyte cell line grown in organotypic culture have been observed.

To observe more detailed characteristics of the parental (passage 5) and NIKS® (passage 38) organotypic cultures and to confirm the histological observations, samples were analyzed using electron microscopy. Parental cells and the immortalized NIKS® human keratinocyte cell line were harvested after 15 days in organotypic culture and sectioned perpendicular to the basal layer to show the extent of stratification. Both the parental cells and the NIKS® cell line undergo extensive stratification in organotypic culture and form structures that are characteristic of normal human epidermis. Abundant desmosomes are formed in organotypic cultures of parental cells and the NIKS® cell line. The formation of a basal lamina and associated hemidesmosomes in the basal keratinocyte layers of both the parental cells and the cell line was also noted.

Hemidesmosomes are specialized structures that increase adhesion of the keratinocytes to the basal lamina and help maintain the integrity and strength of the tissue. The presence of these structures was especially evident in areas where the parental cells or the NIKS® cells had attached directly to the porous support. These findings are consistent with earlier ultrastructural findings using human foreskin keratinocytes cultured on a fibroblast-containing porous support. Analysis at both the light and electron microscopic levels demonstrate that the NIKS® cell line in organotypic culture can stratify, differentiate, and form structures such as desmosomes, basal lamina, and hemidesmosomes found in normal human epidermis.

In some embodiments, the tissues that are supported on the porous membrane and enclosed with the container assembly are cryopreserved. Where this tissue is a skin equivalent, the cryopreserved skin equivalents are preferably storable at approximately −50 C, −60 C, −70 C, −80 C or colder for an extended period of time such as greater than 1, 2, 3, 4, 5 or 6 months and up to 12 or 24 months without a substantial loss of viability.

In preferred embodiments, all steps of the cryopreservation process prior to product packaging are performed aseptically inside a Class 100 biosafety cabinet in a Class 10,000 cleanroom. In some embodiments, the cryopreservation process comprises treating an organotypically cultured skin equivalent in a cryoprotectant solution. The organotypically cultured skin equivalent is supported on a porous membrane of a tray of the present disclosure, and the tray is placed in a suitable vessel, such as a culture vessel or a tissue container assembly of the present disclosure. A suitable volume of cryoprotectant solution is added to the vessel to be in contact with the porous membrane, but not submerge the tissue, allowing cryoprotectant transfer into the tissue through its base. Certain embodiments of the present invention are not limited to the use of any particular cryoprotectant. In some preferred embodiments, the cryoprotectant is glycerol. The cryoprotectant may be provided in different concentrations in the cryoprotectant solution. In some embodiments, the cryoprotectant is provided in a solution comprising about 20% or 21% to about 70% of the solution by volume, and more preferably about 20% or 21% to about 45% of the solution by volume or 37.5% to 62.5% of the solution by volume, or most preferably from about 25% to 40% of the solution by volume or 42.5% to 57.5% of the solution by volume, depending on the temperature. In some embodiments, the cryoprotectant solution preferably comprises about 32.5% v/v or about 50% v/v cryoprotectant (e.g., glycerol). In some embodiments, the cryoprotectant is provided in a base medium solution. Suitable base medium solutions include, but are not limited to, DMEM, Ham's F-10, Ham's F-12, DMEM/F-12, Medium 199, MEM and RPMI. In some embodiments, the base medium forms the remainder of the solution volume. In some embodiments, the cryoprotectant solution is buffered. Suitable buffers include, but are not limited to, HEPES, Tris, MOPS, and Trizma buffers. Buffering agents may be included at an amount to provide a buffered system in the range of pH 7.0 to 7.4. In some preferred embodiments, the cryoprotectant solution is buffered with from about 5 mM to 15 mM HEPES, most preferably about 10 mM HEPES to a pH of about 7.0 to 7.4.

In some particularly preferred embodiments, treatment with the cryoprotectant solution is conducted in a single step. By “single step” it is meant that the cryoprotectant solution is not exchanged during the equilibration procedure as is common in the art. For example, the treatment step is performed using a cryoprotectant solution with a defined concentration of cryoprotectant as opposed to a stepwise equilibration procedure where several media changes with increasing concentrations of cryoprotectant at each step. In some embodiments, the treatment step is conducted at a reduced temperature. In preferred embodiments, the treatment step is conducted at from about 2 C to 8 C, while in other embodiments, the treatment step is conducted at room temperature, for example from about 15 C to 30 C. In some embodiments, the skin equivalent is incubated in the cryoprotectant solution for about 10 to 60 minutes, preferably from about 20 to 30 minutes.

In some embodiments, the skin equivalent supported on the porous membrane of the tray is frozen following treatment with the cryoprotectant solution, preferably after excess cryoprotectant solution is removed from the skin equivalent, for example by aspirating the solution or moving the treated skin equivalent to a fresh vessel (e.g., a sterile culture vessel or a sterile tissue container assembly of the present disclosure). Accordingly, in some embodiments, the treated skin equivalent supported on the porous membrane of the tray is frozen by exposure to temperatures ranging from about −50 C to −100 C, and most preferably at about −80 C. In some preferred embodiments the tray with the treated skin equivalent is simply placed in a bag or other vessel (e.g., a sterile culture vessel or a sterile tissue container assembly of the present disclosure) and placed in a freezing unit such as a low temperature (e.g., −80° C. freezer) freezing unit. In contrast, it is common in the art to control the rate of freezing either by controlling the temperature in the freezing unit or by placing the tissue to be frozen in a container that allows control of the rate of decrease in temperature.

In some embodiments, the cryopreserved skin equivalent is packaged for long term storage. In some preferred embodiments, the skin equivalent, in its tray, is enclosed with the bottom and top containers as described in detail above. In some embodiments, the assembly containing the human skin equivalent is sealed, preferably heat sealed in a sterile bag (e.g., a plastic, foil, or polymer bag) to provide a primary package. In some embodiments, the primary package is then sealed inside a secondary bag, for example a secondary plastic, foil, or Mylar bag. In other embodiments, the primary package may be a laminated, foil pouch. The cryopreserved tissues of the present invention may preferably be stored at a low temperature, from about −50 C to about −100 C or lower, preferably about −80 C. The skin equivalents may be preferably stored from about 1, 2, 3, 4, 5 or 6 months and up to 12 or 24 months without a substantial loss of viability.

In a preferred embodiment, an organotypically cultured skin equivalent in its tray, which is inserted into a sterile bottom container of the present disclosure, is treated with a cryoprotectant solution as described above. Excess cryoprotectant solution is removed from the skin equivalent prior to freezing by aspirating the cryoprotectant solution from the bottom container. The treated skin equivalent in its tray is then enclosed with a sterile top container of the present disclosure, thereby forming a tissue container system. Alternatively, excess cryoprotectant solution is removed from the skin equivalent prior to freezing by moving the tray with the treated skin equivalent to a second, sterile bottom container of the present disclosure and then enclosing the tray with a sterile top container of the present disclosure, thereby forming a tissue container system. The tissue container system containing the treated human skin equivalent is then sealed, preferably heat sealed in a sterile bag (e.g., a plastic, foil, or polymer bag) to provide a primary package. In some embodiments, the primary package may be sealed inside a secondary bag, for example a secondary plastic, foil, or Mylar bag. In other embodiments, the primary package may be a laminated, foil pouch. The primary or secondary bag is then stored at low temperature, from about −50 C to about −100 C, preferably about −80 C. The skin equivalents may be stored from about 1, 2, 3, 4, 5 or 6 months and up to 12 or 24 months without a substantial loss of viability.

In another preferred embodiment, an organotypically cultured skin equivalent in its tray, which is placed in a culture vessel, is treated with a cryoprotectant solution as described above. Excess cryoprotectant solution is removed from the skin equivalent prior to freezing by moving the tray with the treated skin equivalent to a sterile bottom container of the present disclosure and then enclosing the tray with a sterile top container of the present disclosure, thereby forming a tissue container system. The tissue container system containing the treated human skin equivalent is then sealed, preferably heat sealed in a sterile bag (e.g., a plastic, foil, or polymer bag) to provide a primary package. In some embodiments, the primary package may be sealed inside a secondary bag, for example a secondary plastic, foil, or Mylar bag, to produce a secondary package. In other embodiments, the primary package may be a laminated, foil pouch. The primary or secondary package is then stored at low temperature, from about −50 C to about −100 C, preferably about −80 C. The skin equivalents may be stored from about 1, 2, 3, 4, 5 or 6 months and up to 12 or 24 months without a substantial loss of viability.

In some embodiments, the present invention provides a method of thawing a cryopreserved skin equivalent prior to application to a subject, comprising providing a cryopreserved tissue in the tissue container system as described above, removing the top tissue container to expose the cryopreserved tissue, and filling the reservoir in the bottom tissue container with a liquid medium under conditions that the cryopreserved tissue thaws to provide a thawed tissue, where the cryoprotectant contained within the tissue is diluted into the liquid medium, leaving a tissue that is substantially free of cryoprotectant. In other embodiments, the method comprises removing a primary or secondary package containing a tissue container system comprising a cryopreserved tissue from a freezer or shipping container, removing the tissue container system from the package(s), removing the top tissue container to expose the cryopreserved tissue, and transferring the tray with the cryopreserved skin equivalent from the first tissue container into a second tissue container that is sterile and staged in the sterile field and contains a liquid medium in the container reservoir, such that the transferred cryopreserved tissue thaws to provide a thawed tissue and the cryoprotectant contained within the tissue is diluted into the liquid medium. In some of the above embodiments, the liquid medium is a tissue compatible solution, preferably a buffered solution. Suitable tissue compatible solutions include, but are not limited to, DMEM, Ham's F-10, Ham's F-12, DMEM/F-12, Medium 199, MEM and RPMI. Suitable buffers include, but are not limited to, HEPES, Tris, MOPS, and Trizma buffers. Buffering agents may be included at an amount to provide a buffered system in the range of pH 7.0 to 7.4. In still other embodiments, the tray with the cryopreserved skin equivalent is removed from the tissue container and placed on an absorbent medium to remove thawed cryoprotectant solution from the skin equivalent. The absorbent medium may be in any suitable, preferably sterile, vessel (e.g., a culture vessel or a fresh tissue container assembly). The present invention is not limited to the use of a particular absorbent medium. Suitable absorbent media include, but are not limited to, Telfa® pads, cellulosic pads (e.g., Whatman 1003-090 filter pads and Pall 70010 filter pads), gauze pads, and foam pads (e.g., Covidien 55544 hydrophilic foam pad). In some preferred embodiments, the absorbent medium is a Telfa® pad. In some embodiments, the absorbent medium further comprises a tissue-compatible solution. In some embodiments, the tissue compatible solution is a buffered solution. Suitable tissue compatible solutions include, but are not limited to, DMEM, Ham's F-10, Ham's F-12, DMEM/F-12, Medium 199, MEM and RPMI. Suitable buffers include, but are not limited to, HEPES, Tris, MOPS, and Trizma buffers. Buffering agents may be included at an amount to provide a buffered system in the range of pH 7.0 to 7.4.

It is contemplated that the cryopreserved skin equivalents of the present invention may be used therapeutically after thawing. In some embodiments, the cryopreserved skin substitute is used after thawing in wound closure and burn treatment applications. The use of autografts and allografts for the treatment of burns and wound closure is described in Myers et al., A. J. Surg. 170(1):75-83 (1995) and U.S. Pat. Nos. 5,693,332; 5,658,331; and 6,039,760, each of which is incorporated herein by reference. In some embodiments, the skin equivalents may be used in conjunction with dermal replacements such as DERMAGRAFT or INTEGRA. Accordingly, the present invention provides methods for wound closure, including ulcers or wounds caused by burns, comprising providing a cryopreserved skin equivalent in a tissue container system of the present disclosure, thawing the skin equivalent, and treating a patient suffering from a wound with the thawed skin equivalent under conditions such that the wound is closed.

In some embodiments, the skin equivalents are utilized to treat chronic skin wounds. Chronic skin wounds (e.g., venous ulcers, diabetic ulcers, pressure ulcers) are a serious problem. The healing of such a wound often takes well over a year of treatment. Treatment options currently include dressings and debridement (use of chemicals or surgery to clear away necrotic tissue), and/or antibiotics in the case of infection. These treatment options take extended periods of time and high levels of patient compliance. As such, a therapy that can increase a practitioner's success in healing chronic wounds and accelerate the rate of wound healing would meet an unmet need in the field. Accordingly, the present invention contemplates treatment of skin wounds with cryopreserved skin equivalents. In some embodiments, skin equivalents are topically applied to wounds after thawing. In other embodiments, cryopreserved skin equivalents are used for application to partial thickness wounds after thawing. In other embodiments, cryopreserved skin equivalents are used to treat full thickness wounds after thawing. In other embodiments, cryopreserved skin equivalents are used to treat numerous types of internal wounds after thawing, including, but not limited to, internal wounds of the mucous membranes that line the gastrointestinal tract, ulcerative colitis, and inflammation of mucous membranes that may be caused by cancer therapies. In still other embodiments, skin equivalents expressing host defense peptides or pro-angiogenic factors are used as a temporary or permanent wound dressing after thawing.

In still further embodiments, the cells are engineered to provide additional therapeutic agents to a subject. The present invention is not limited to the delivery of any particular therapeutic agent. Indeed, it is contemplated that a variety of therapeutic agents may be delivered to the subject, including, but not limited to, enzymes, peptides, peptide hormones, other proteins, ribosomal RNA, ribozymes, small interfering RNA (siRNA) micro RNA (miRNA), and antisense RNA. In preferred embodiments, the agents are host defense peptides such as human beta-defensin 1, 2, or 3 or cathelicidin or other proteins such as VEGF and HIF-1 a, see, e.g., U.S. Pat. Nos. 7,674,291; 7,807,148; 7,915,042; 7,988,959; and 8,092,531; each of which is incorporated herein by reference in its entirety. These therapeutic agents may be delivered for a variety of purposes, including but not limited to the purpose of correcting genetic defects. In some particular preferred embodiments, the therapeutic agent is delivered for the purpose of detoxifying a patient with an inherited inborn error of metabolism (e.g., aminoacidopathesis) in which the skin equivalent serves as wild-type tissue. It is contemplated that delivery of the therapeutic agent corrects the defect. In some embodiments, the cells are transfected with a DNA construct encoding a therapeutic agent (e.g., insulin, clotting factor IX, erythropoietin, etc.) and skin equivalents prepared from transfected cells are administered to the subject. The therapeutic agent is then delivered to the patient's bloodstream or other tissues from the graft. In preferred embodiments, the nucleic acid encoding the therapeutic agent is operably linked to a suitable promoter. The present invention is not limited to the use of any particular promoter. Indeed, the use of a variety of promoters is contemplated, including, but not limited to, inducible, constitutive, tissue-specific, and keratinocyte-specific promoters. In some embodiments, the nucleic acid encoding the therapeutic agent is introduced directly into the keratinocytes (i.e., by electroporation, calcium phosphate co-precipitation, or liposome transfection). In other preferred embodiments, the nucleic acid encoding the therapeutic agent is provided as a vector and the vector is introduced into the keratinocytes by methods known in the art. In some embodiments, the vector is an episomal vector such as a replicating plasmid. In other embodiments, the vector integrates into the genome of the keratinocytes. Examples of integrating vectors include, but are not limited to, retroviral vectors, adeno-associated virus vectors, non-replicating plasm id vectors and transposon vectors

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); mM (millimolar); μM (micromolar); N (Normal); mol (moles); mmol (millimoles); μmol (micromoles); nmol (nanomoles); g (grams); mg (milligrams); lig (micrograms); ng (nanograms); 1 or L (liters); ml or mL (milliliters); μ1 or μL (microliters); cm (centimeters); mm (millimeters); pm (micrometers); nm (nanometers); C (degrees Centigrade); U (units), mU (milliunits); min. (minutes); sec. (seconds); % (percent); kb (kilobase); bp (base pair); PCR (polymerise chain reaction); BSA (bovine serum albumin); CFU (colony forming units); kGy (kiloGray); PVDF (polyvinylidine fluoride); BCA (bicinchoninic acid); SDS-PAGE (sodium dodecyl sulfate polyacrylamide gel electrophoresis).

### Example 1

StrataGraft® skin tissue is a living, full-thickness, allogeneic human skin substitute that reproduces many of the structural and biological properties of normal human skin. StrataGraft® skin tissue contains both a viable, fully-stratified epidermal layer derived from NIKS® cells, which are a consistent and well-characterized source of pathogen-free human keratinocyte progenitors, and a dermal layer containing normal human dermal fibroblasts (NHDF) embedded in a collagen-rich matrix. StrataGraft® skin tissue possesses excellent tensile strength and handling characteristics that enable it to be meshed, stapled, and sutured similarly to human skin grafts. StrataGraft® also exhibits barrier function comparable to that of intact human skin and is capable of delivering bioactive molecules for wound bed conditioning and tissue regeneration. The physical and biological characteristics of StrataGraft® skin tissue make it ideal for the treatment of a variety of skin wounds.

The manufacturing process for StrataGraft® skin tissue encompasses three sequential cell and tissue culture processes. In Stage I of the manufacturing process, NIKS® keratinocytes are expanded in monolayer cell culture. Concurrent with the NIKS® keratinocyte culture in Stage I, NHDF are expanded in monolayer culture and combined with purified type I collagen and culture medium and allowed to gel to form the cellularized dermal equivalent (DE). Alternatively, NHDF are seeded into Transwell® inserts (Corning) and allowed to proliferate and secrete and assemble extracellular matrix molecules into a simplified dermal equivalent. In Stage II, NIKS® keratinocytes are seeded onto the surface of the DE and cultured under submerged conditions for two days to promote complete epithelialization of the DE surface. The tissue is then lifted to the air-liquid interface in Stage III, where it is maintained for 18 days in a controlled, low humidity environment to promote tissue maturation. The skin equivalents are generally prepared as described in U.S. Pat. Nos. 7,674,291; 7,807,148; 7,915,042; 7,988,959; 8,092,531; and U.S. Pat. Publ. 20140271583; each of which is incorporated herein by reference in its entirety.

### Example 2

This example describes improved cryopreservation methods for human skin equivalents utilizing a pre-freeze treatment step with cryopreservation solutions containing 32.5% or 50% glycerol at room temperature and is described in copending U.S. Pat. Publ. 20140271583, which is incorporated by reference herein in its entirety. The general production process is unchanged from the current method described previously. At the end of the production process, the tissues are treated and cryopreserved as follows.

| Parameter | Operating Range |
|---|---|
| Cryoprotectant formulation | 32.5% (v/v) glycerol |
| | DMEM (1X) |
| | 10 mM HEPES (pH 7.0 to 7.4); or |
| | 50% (v/v) glycerol |
| | DMEM (1X) |
| | 10 mM HEPES (pH 7.0 to 7.4) |
| Pre-freeze cryoprotectant | Room temperature |
| incubation temperature | |
| Pre-freeze cryoprotectant incubation time | 15-45 minutes |
| Freeze method | Direct transfer to −80 C. freezer |
| Storage temperature | −70 to −90 C. |
| Shipping conditions | Overnight delivery on dry ice |

All steps of the cryopreservation process prior to the final product packaging step are performed aseptically inside a Class 100 biosafety cabinet in a Class 10,000 cleanroom. The specific volumes and dishes described in this example are applicable to tissues generated in the previous circular, 44 cm² format, not the larger rectangular format of the current disclosure.

Step 1—Dispense 20 ml of cryoprotectant solution to 100 mm culture dishes.

Step 2—Transfer Transwell® inserts containing StrataGraft® tissues into individual dishes containing cryoprotectant solution. Incubate tissues 15-45 minutes in cryoprotectant solution.

Step 3—Transfer Transwell® inserts containing treated StrataGraft® tissues to new sterile 100 mm culture dishes containing final product label so that the tissue rests on the bottom of the culture dish. Excess cryoprotectant is allowed to drain from the skin equivalent to provide a treated skin equivalent that is substantially free of excess cryoprotectant on the exterior surfaces of the skin equivalent.

Step 4—Heat-seal 100 mm culture dishes in clear, sterile bags. Place primary package into secondary Mylar bag and heat-seal.

Step 5—Remove the packaged StrataGraft® tissues from cleanroom and transfer tissues to an ultralow freezer (−70° C. to −90° C.). Place tissues in a pre-cooled rack in the freezer that allows unrestricted airflow to the top and bottom of the packaged tissues to ensure uniform and rapid cooling. Leave tissues undisturbed overnight during the freezing process.

Cryopreserved tissues were thawed at room temperature for 10 minutes, transferred to a hold chamber containing Telfa® pads saturated with 40 ml of HEPES-buffered culture medium that had been warmed to room temperature (RT), and held at RT for 15 to 20 minutes. Tissues were transferred to a culture dish containing 90 ml of SMO1 medium and returned to culture overnight. Tissues were analyzed for viability after overnight re-culture. Tissues treated with 32.5% glycerol at room temperature for 15 to 45 minutes had acceptable post-thaw viability. Tissues treated with 50% glycerol at room temperature for 15 minutes also had acceptable viability; however, tissues treated with 50% glycerol at room temperature for 45 minutes had unacceptable viability.

### Example 3

This study was performed to evaluate the performance of product packaging plasticware, which is a tissue container assembly of the present disclosure, for use as packaging for cryopreserved StrataGraft® tissues. The study evaluated three independent lots of rectangular, 100 cm² StrataGraft® tissues comparing tissues packaged in the Transwell® growth chamber and those packaged in the tissue containers described herein. For each batch, post-thaw properties of tissues packaged in the tissue containers of the instant invention were evaluated following different hold conditions and compared to those of control tissues using current packaging and thaw/hold procedures. The results of this study demonstrated that tissue containers of the instant invention are suitable for use in transporting and thawing cryopreserved StrataGraft® tissues and that acceptable thawing can be achieved in the sterile field without use of a Telfa® pad.

StrataGraft® skin tissues are produced in batches of 100 cm² StrataGraft® skin tissues. This larger tissue format and increase in batch sizes put an added emphasis on efficient storage and shipment of the skin tissues. To address that issue, plasticware tissue containers were designed which reduce the volume of the final packaged product by 60% compared to packaging in the Transwell® growth chamber as disclosed in copending U.S. Pat. Publ. 20140271583. In this example, this packaging is introduced into the process following cryoprotectant treatment, immediately before the product is sealed in the foil pouch and transferred to an ultracold freezer for long-term storage. The tissue containers of the instant invention were designed with a 0.75 mm deep reservoir below tissue that can be flooded with hold solution. This design allows the packaging to be used as a post-thaw hold container, which simplifies the preparation of StrataGraft® tissue for clinical use by eliminating the need for a separate hold basin.

This experiment evaluated the post-thaw properties of StrataGraft® skin tissues from three batches, and frozen in either a Transwell® growth chamber or in the tissue containers of the instant invention. In addition, this study evaluated post-thaw hold procedures performed in the tissues containers of the instant invention without the use of Telfa® pads, compared to control hold conditions performed in basins containing Telfa® pads.

| | Pre-Freeze | | Thaw | Hold | | Hold |
|---|---|---|---|---|---|---|
| Group | Treatment | Packaging | Condition | Chamber | Hold Solution | Condition |
| 1 | 37.5% | Transwell ® | 10 min at | DeRoyal | 250 mL Hold | 15-20 min |
| | glycerol | Growth | RT | Basin | Solution | at RT |
| | 20 min at RT | Chamber | | (2-Telfa) | Warmed to 35-39° C. | |
| 2 | | Tissue | | | | |
| 3 | | container | | Tissue | 15 mL Hold | |
| | | assembly | | container | Solution | |
| | | | | assembly (no | Warmed to 35-39° C. | |
| | | | | Telfa ®) | | |

Batches of 20 rectangular, 100 cm² StrataGraft® skin tissues were produced using standard processes. Briefly, NIKS® cells and normal human dermal fibroblasts (NHDF) were expanded in monolayer culture. NHDF were thawed and expanded in monolayer. Following expansion, the NHDF cells were harvested and mixed into a type I collagen solution, dispensed to 100 cm² rectangular trays of the present disclosure (tissue-culture treated polycarbonate membrane, nominal thickness of about 10 microns, nominal pore size of about 0.4 microns), and gelled to create the dermal equivalent layer (DE). After gelling, the DE was submerged in media in a growth chamber and cultured for five days prior to the NIKS® seed. NIKS® were thawed, expanded, and then harvested and seeded onto DE surfaces. Tissues were maintained in submerged culture for two days to allow for attachment and proliferation of NIKS® over the DE surface and then cultured at the air-liquid interface for 18 days to enable complete epidermal differentiation. Transfers of media, NHDF/collagen mixture, and NIKS® suspension to the trays and Transwell® growth chambers were performed using peristaltic pumps.

At the end of the production process, culture media was aspirated and tissues were treated in the Transwell® growth chamber with 50 mL of cryopreservation solution containing 37.5% glycerol for 20 minutes at room temperature (RT) whilst still supported on the membrane of the tray. At the end of treatment, the trays containing the nine tissues designated for this experiment were removed from the excess cryopreservation solution and packaged into one of two packaging configurations: 1) three tissues were kept in the Transwell® growth chamber in the high position and sealed inside of 7.875″×12″ foil pouches (Group 1); and 2) six tissues were transferred to sterile tissue containers of the instant invention and sealed inside 6.75″×10.25″ foil peel pouches (Group 2 and Group 3, n=3 per group). At the end of packaging, all packaged tissues were transferred to an ultracold freezer and stored at −70 to −90° C. until analysis.

Group 1 and Group 2 tissues were then thawed using previously established procedures that utilized an absorbent medium (e.g., Telfa® pad). Group 3 tissues were thawed using a simplified hold procedure. Briefly, Group 3 cryopreserved tissues were thawed at room temperature for 10 minutes in the tissue container in which the tissue was frozen, the bottom tissue containers were then flooded with hold solution (15 ml of HEPES-buffered culture medium that had been warmed to 35-39 C) and held at room temperature for 15 to 20 minutes. Following the post-thaw hold, tissues from all groups were transferred to new rectangular growth chambers containing SMO1 and re-cultured for 22 to 26 hours

Tissues were evaluated for appearance, barrier function, viability, histology, and VEGF secretion in the conditioned media. In addition, whole tissue MTT staining was performed to evaluate uniformity of the tissue viability. The results of tissues frozen in the tissue containers of the instant invention (groups 2 and 3) were compared to those of the control group.

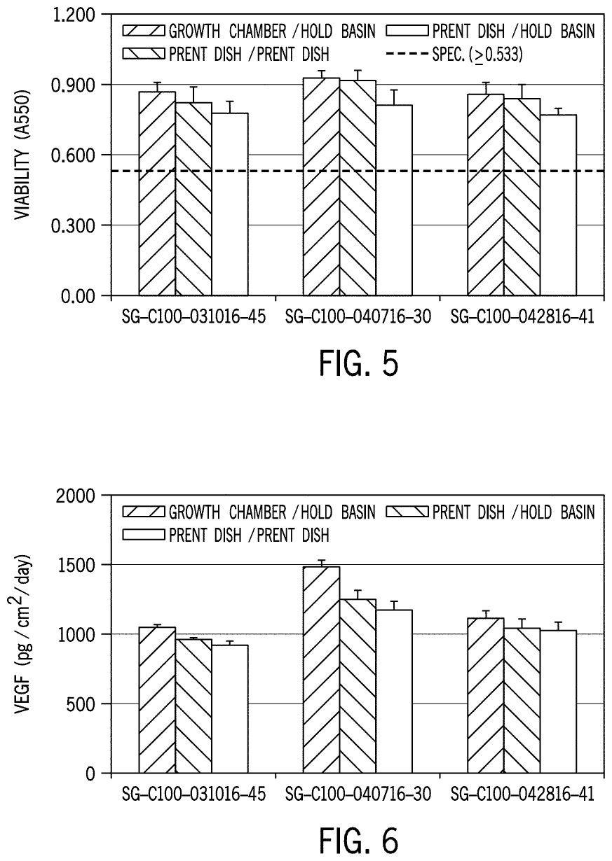

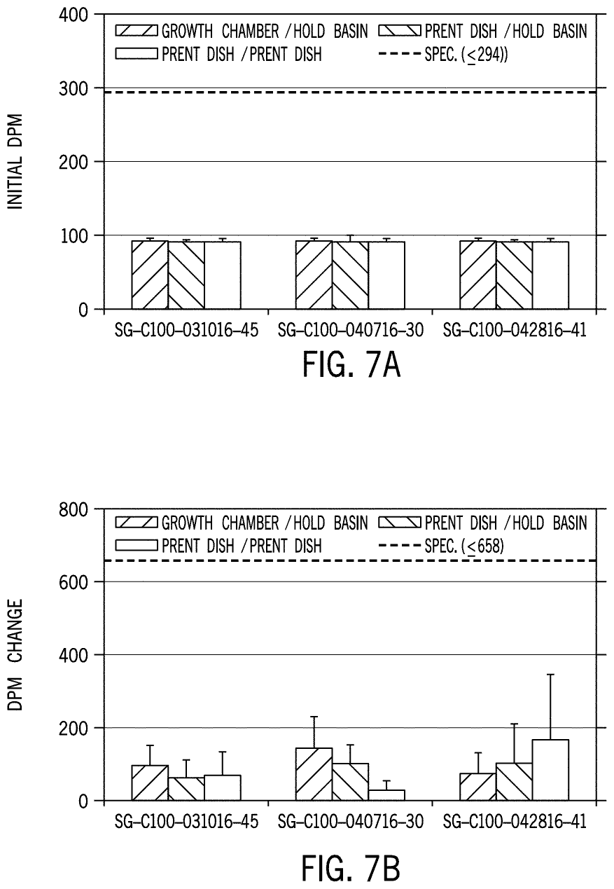

The results of this study demonstrate that use of the tissue containers of the instant invention does not affect the properties of cryopreserved StrataGraft® tissues. Tissues packaged in the two configurations and thawed/held using the previously established procedures (Groups 1 and 2) had comparable appearance, histology, viability, and barrier function, and VEGF secretion. The tissue containers of the instant invention also showed promising results for use in a simplified hold procedure. Tissues packaged and kept in tissue containers of the instant invention for the post-thaw hold (Group 3) had similar properties to both other groups. Tissue appearance, histology, VEGF secretion, and barrier function were not significantly different than control tissues (Group 1); viability showed a modest (−10%), but statistically significant (p<0.05), reduction compared to controls, while still easily exceeding the established lot release criterion. MTT staining patterns of tissues from all groups were comparable, with qualitatively consistent staining across the tissue surfaces. See FIGS. 5, 6 and 7.

### Example 4

The allogeneic cellularized scaffold product was prepared by the following steps:

The allogeneic cellularized scaffold product was provided in a product dish enclosed in a foil pouch and a carton. Within the foil pouch, the allogeneic cellularized scaffold product was contained in a polystyrene tray and loosely adhered to a polycarbonate membrane contained within the polystyrene tray. The polystyrene tray was contained within the product dish. A hold solution was provided in a plastic bottle contained in a laminated, foil pouch. A holding dish was provided in a clear pouch consisting of a top portion and a bottom portion.

The hold solution was then removed from the laminated, foil pouch. The hold solution was then placed in a warming oven was used to warm the hold solution to 35-39° C. for at least 45 minutes prior to use, or in a water bath for at least 15 minutes prior to use. When the water bath was used, the cap or threads of the bottle were not submerged in the bath. An operator then peeled open the seal of the clear pouch containing the hold dish, which was then removed aseptically from the pouch and placed in a sterile field by another operator.

The allogeneic cellularized scaffold product was then removed from the carton. The foil pouch was then peeled open and the polystyrene tray was removed and placed on a nonsterile surface.

The hold solution was removed from the warming oven and immediately poured into the sterile hold dish using aseptic technique. A nonsterile operator then removed the lid from the product dish without contacting the polystyrene insert tray. A sterile operator then aseptically removed the polystyrene insert tray from the product dish using either sterile, gloved fingers or forceps. The sterile operator then placed the polystyrene tray into the hold dish, beginning with one edge and lowering it to the opposite edge to minimize trapping bubbles beneath the insert tray. If bubbles were trapped beneath the insert tray, the sterile operator gently lifted the insert tray and placed it slowly back down in the hold solution. The insert tray containing the allogeneic cellularized scaffold product was then maintained in the hold solution for at least 15 minutes, but no longer than 4 hours.

Last, the allogeneic cellularized scaffold product was removed from the polycarbonate membrane using sterile, gloved fingers or a pair of atraumatic forceps. The allogeneic cultured keratinocyte composition was then meshed up to a ratio of 1:1. The allogeneic cultured keratinocyte composition was not allowed to dry: the mesher and tissue board were moistened as needed using hold solution, sterile 0.9% normal saline, or lactated Ringer's solution to prevent drying.

### Example 5

Oxygen and moisture permeability studies were conducted on the product packaging in accordance with ASTM guidance's due to the lack of applicable testing in the USP for cellular therapy and tissue products. In order to assess the oxygen transmission of the skin tissue packaging, testing was performed on the foil pouch material used to package the skin tissue in accordance with ASTM D3985. Testing results demonstrated that oxygen transmission was <0.003 cc/100 in²/24 hours. Additional testing was done in accordance with ASTM F1249 to assess the moisture permeability of the pouch. Results demonstrated moisture permeation of <0.0065 g/100 cm²/24 hours. The results support that the skin tissue foil pouch can provide an adequate barrier from oxygen and moisture for the skin tissue.

The primary barrier from microbiological contamination is the pouch, thus this component was assessed for seal integrity. Container closure integrity was also validated. Additionally, the seal integrity was demonstrated through sterility testing at the time of release and as part of the stability program.

### Example 6

In accordance with USP<661> Biological Tests—Plastics and Other Polymers, the Biological Reactivity tests were conducted according to USP<87> Biological Reactivity Tests, In Vitro. Either the agar diffusion or elution test was performed on representative gamma sterilized components (tissue insert tray and product dish). The test articles were considered non-cytotoxic and met the USP<87> requirements. USP<88> Biological Reactivity testing was performed on the product dish components. Results were as follows.

| Sample (Materials) | Results |
|---|---|
| Tissue Insert - 100 cm² membrane | Non-cytotoxic |
| | Meets requirements of USP<87> Elution Test |
| Tissue Insert - Polystyrene Frame | Non-cytotoxic |
| | Meets requirements of USP<87> Elution Test |
| Product Dish | Non-cytotoxic |
| | Meets requirements of USP<88> Class VI Testing |

As the skin tissue is a living, biological product manufactured in complex culture media and containing collagen and viable human cells, traditional analysis of chemical leachables using the final product was not feasible. The copious number of large- and small-molecular weight compounds and likely presence of interfering substances in the final product would severely limit the ability to detect and identify leachables in the final product. Thus, as part of the manufacturing and packaging component qualification, analytical methods were developed that allow for the quantification of leachable compounds within the media components that come into contact with the skin tissue during manufacture and long term storage.

The growth chamber assembly, used during manufacture of the skin tissue, consisted of two component parts: 1) the tissue insert and 2) the growth chamber, which consisted of a culture reservoir and lid that supported and enclosed the tissue insert, as shown below.

| Component | Aspect | Specification |
|---|---|---|
| Tissue Insert Frame | Material | Injection-molded polystyrene |
| | | (PS) |
| Tissue Insert Membrane | Material | Polycarbonate (PC) |
| | Nominal pore density | 1 × 10⁸ pores per cm² |
| | Nominal pore size | 0.4 μm |
| | Nominal thickness | 10 μm |
| Tissue Insert (frame and | Surface treatment | Tissue-culture (plasma) treated |
| membrane) | | |
| Growth Chamber and Lid | Material | Thermoformed polyethylene |
| | | terephthalate, glycol-modified |
| | | (PETG) |
| Tissue Insert (all components) | Sterility | Gamma irradiated |

Collectively, the growth chamber and tissue insert comprised the Growth Chamber Assembly. StrataGraft tissue production involved 25 days of culture in the Growth Chamber Assembly at 37±2° C. in a humidified, 5% CO₂ incubator. During this period, the collagen and cells were retained in the 100 cm² tissue insert tray, which constrained and defined the tissue size and shape. The porous tissue insert membrane supported the tissue throughout development and allowed nutrient and waste exchange with culture medium, which was composed of SM (Stratification Media) base media plus media supplements, in the growth chamber reservoir. Media was fed into the tissue insert interior (for a portion of the culture period) and growth chamber reservoir (for the entire period), with periodic media changes during manufacturing.

All component materials used to manufacture the growth chamber and tissue insert assembly were tested using USP <87> (in vitro biological reactivity) and/or USP <88> (in vivo biological reactivity) and were found to exhibit no cytotoxicity and met Class VI standards, respectively. Additionally, these materials had no noticeable adverse effects on the tissue or its cellular components during the lengthy organotypic culture process. The manufacturing components, comprised of the tissue growth chamber and tissue insert, were assessed for both extractables and leachables.

The product dish/hold dish packaging components were also evaluated. Prior to use, StrataGraft skin tissues were held within sterile hold dishes identical in design to the product dishes that were part of the packaging. Each of these components were tested in extractables studies using 20% ethanol in water, as well as leachables studies using the base media that contacts the components during their manufacture, storage, and clinical use. Additionally, an extractables analysis using hexane was performed for the product dish/hold dish packaging components. Each of the components was tested in the absence of the scaffold product to prevent analytical interference by growth factors, cytokines, peptides, and small molecules secreted by the viable skin tissue during manufacture. The following compounds were tested for as lechates: caffeine, nitrobenzene-d5, acenaphthene-d10, diphenylamine, hexamethoxyphosphazene, pentadecanoic acid, hexakis(2,2,3,3-tetrafluoropropoxy) phosphazene, pentachlorophenol, and di(piperidin-1-yl) methanethione.

Two parallel studies were performed using either 20% ethanol/80% water as the extraction solvent and SM base medium as the solvent for leachable assessment.

Cramer class III guidelines were used in this study to define the AET, which would allow up to 90 μg of an unknown compound to be delivered during a single application of StrataGraft skin tissue. For the purposes of the extractable leachable studies, a maximum application of up to 30 tissues per day was factored into the calculation. Thus, the AET was set to 3 μg per tissue or 3 μg per Growth Chamber and Tissue Insert Assembly.

Analysis by GC-MS and HS-GC-MS did not yield any unknown compounds in the 20% ethanol samples or the SM base media samples above the AET. HPLC-UV-MS analysis on the 20% ethanol samples and SM base media samples did not yield any unknown compounds above the AET. While not exceeding the AET, two unknown compounds were detected in the 20% ethanol samples in the positive ion mode (no unknown compounds in the negative ion mode), which were over 1.5 μg/Assembly. Tentatively, the molecular formulas associated with unknown compound 1 and 2 were C₁₁H₂ON₂ and C₈H₁₄N₂O, respectively. Importantly, in samples using the SM base media used in the StrataGraft manufacturing process, all leachable compounds fell below assay limit of detection.

When assessing the extractable and leachable elemental impurities using the ICP-MS method, most elements were either not detected (ND) or below the LOQ (Limit of Quantitation) for the method. If the response for an element in the sample was less than or equal to the response for that element in the control, then the result reported was not detected (ND). Boron (B) and aluminum (Al) were detected at sub-ppm range in the extractable study, however only Boron was detected in the leachable study. Tin was detected at low level in one replicate during the leachable study, however the detected level of tin does not pose a risk to patients receiving a dosage of 30 StrataGraft skin tissues as the levels are below the permitted daily allowance limits.

### Example 7

At the end of the manufacturing process, mature StrataGraft tissues were treated in their growth chambers with a cryoprotectant (CPS). Following that treatment, the tissues, in their tissue inserts, were transferred to sterile product dishes, which were then sealed inside laminated foil peel pouches. The packaged tissues were held briefly, for up to 30 minutes, at room temperature during the packaging period and then transferred to ultra-cold freezers to complete cryopreservation. Tissues were maintained at −70 to −90° C. during storage and transported on dry ice.

Prior to use, cryopreserved StrataGraft skin tissues were thawed and held in an aqueous buffered hold solution to dilute the CPS present in the tissue. Thaw and hold procedures were optimized to provide flexibility for late-stage clinical and commercial use. Prior to use, hold solution bottles were pre-warmed to 35 to 39° C. and then the contents (15 mL) were poured into sterile hold dishes (identical in design to the product dishes that were part of the packaging) staged in the sterile field. StrataGraft tissues were removed from cold storage, unpackaged, and the inserts containing the tissues were aseptically transferred into the sterile field and placed in hold dishes containing the hold solution. Tissues were held in the solution for at least 15 minutes to allow adequate dilution of the CPS and can be kept in the dish for up to 4 hours prior to application.

As a result of the transfer of a tissue in the tissue insert to the product dish, a small volume of CPS was transferred to the product dish, comprising the CPS contained within the tissue, as well as CPS on the exterior of the culture insert through surface tension. Extractables and leachables studies were designed to determine and monitor compounds that CPS can extract from the product dish during packaging, storage under ultra-cold conditions, and thawing, and that the hold solution and residual CPS can extract from the hold dish during the hold step before use.

Since the hold dishes and product dishes are identical in design, a single study served as the extractable study for the hold dish as well as the product dish. The insert was previously tested for extractables and leachables as a component of the growth chamber assembly. The AET for that study was calculated using the assumption of 100% delivery of all leachables coming out during the manufacturing process.

Extraction studies were performed with the following extraction solvents and conditions: water at pH 3 with HCl at 37° C. for 24 hours, water at pH 9 with NaOH at 37° C. for 24 hours, hexane at 37° C. for 24 hours, 20% ethanol/80% water at 37° C. for 24 hours, and none (headspace GC-MS) at 90° C. for 40 min.

There were no extractable compounds detected (ND) in samples tested by GC-MS, HPLC-UV-MS, and HS-GC-MS when compared to the controls where anything less than 5% of AET (1.0 μg/mL) was considered as not detected.

The target elements for the ICP-MS analysis were chosen based on USP<232> and ICH Q3D guidelines. Their classification of three classes and their corresponding Permitted Daily Exposure Limits (PDEs) were recommended in the ICH Q3D guideline. Most of the elements were either not detected (ND) or below LOQ. If the response for an element in the sample was less than or equal to the response in the control, then the result reported is non-detected (ND). Sn was detected but well below its respective limit.

B and Al were detected at low ppb levels. For aluminum, WHO has a recommendation not to exceed 1.8 mg/day for an adult from drinking water. As such, the ppb level of aluminum was negligible and does not pose a risk to patients. US EPA has a limit to drinking water of boron at 3 mg/L (assuming children consuming 1 L/per day, while adult consuming 2 L/per day), as such, the daily safety level of boron intake is between 3 mg to 6 mg per day. Therefore, the boron at ppb level in the leachable study is negligible and it does not pose any risk to patients.

### Example 8

This study considered the potential for leachables from the product dish and hold dish. The product dish and hold dish are identical components; however, each of the dishes are exposed to different medium and environmental conditions during long-term storage and clinical use, respectively. The product dish was stored at −70 to −90° C. throughout the shelf life of the drug product. Prior to use, hold solution bottles were pre-warmed to 35 to 39° C. and then poured into sterile hold dishes. During the hold period, a portion of the hold solution moved into the tissue, displacing the cryoprotectant and maintaining the tissue hydration. Once the tissue was removed from the hold dish for clinical use, the described portion of the volume of hold solution was delivered, as part of the product, to the patient upon tissue application. The exposure period of the hold dish to the pre-warmed hold solution was at least 15 minutes and up to 4 hours.

For the evaluation of the product dish, 3 mL of CPS was added to each product dish. The 3 mL CPS exposure approximates the maximum amount of CPS expected to be transferred to the product dish via adherence to the tissue insert during transfer of the StrataGraft skin tissue to the product dish. After adding the CPS, the product dish was sealed in a foil pouch and held for 30 minutes at room temperature. The pouches containing the product dish were then transferred to an ultra-cold freezer for long-term storage at −70 to −90° C. In order to differentiate CPS-related components from leachable compounds, time zero (T0) and control samples consisted of CPS that was stored in sterilized Teflon bottles. The evaluation of leachables was performed at T0, and 3-, 6-, 12-, 18-, 24-, and 36-month time points.

Cramer class III guidelines were used in this study to define the AET, which allows up to 90 μg of an unknown compound per application of StrataGraft skin tissues. For the purposes of the extractable leachable studies, a maximum application of up to 30 tissues per day were factored into the calculation. Thus, the AET was set to 3 μg per tissue or 3 μg per product dish.

A one-time study was performed for the evaluation of the hold dish leachables to simulate clinical-use conditions. Three mL of CPS and 15 mL of hold solution were mixed before adding to each hold dish. The hold dishes containing the mixed medium were conditioned at 37° C. for 4 hours; the maximum allowed duration of the clinical hold. The experiment was performed in duplicate with three hold dishes for each replicate. After conditioning, the test solution consisted of material pooled from the three hold dishes. The controls were prepared the same way as the test samples. Three mL of CPS and 15 mL of hold solution were combined in a glass bottle and conditioned at 37° C. for 4 hours.

A conservative estimate of the volume of the CPS and hold solution mixture, which moves into the tissue, was used to calculate the total amount of a leachable per hold dish that might be delivered to the patient. The fluid volume of a StrataGraft skin tissue should not exceed 2.5 mL, so a maximum of 2.5 mL from the total 18 mL in the hold dish would be contained in the tissue applied to a patient. Therefore, the potential exposure from the Hold Dish is calculated as:

Concentration from the 18 mL of CPS and Hold Solution×2.5 ml per tissue=Total leachable per Hold Dish (μg/dish)

The results from leachables testing of the hold dish containing combined CPS and hold solution were combined with the results from the leachables study evaluating long term storage of the scaffold product in CPS in the product dish. For example, if a leachable compound was detected in both the hold dish study and the long-term storage product dish leachables study, the total exposure was calculated as the sum of the leachable from one product dish and one hold dish. That total was compared to the 3 μg/dish AET.

For the product dish study, there were no leachables from the T=0 control sample by GC-MS and LC-UV-HRMS analyses. For the product dish T=0 CPS control samples, 2-Propanol was detected at 0.7 μg/mL by the HS-GC-MS method. In addition, there were no leachables from the hold dish in the after 4 hours exposure at 37° C. by GC-MS and LC-UV-HRMS analyses. For the hold dish leachables study samples, nothing was detected (non-detected; ND) by HS-GC-MS.

With regards to the ICP-MS analysis, target elements were chosen based on USP<232> and ICH Q3D guidelines. For both product dish long-term storage leachable and hold dish subleachable samples, all tested elements were found below their respective limits with most results being either non-detected (ND) or below LOQ (<LOQ).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in tissue culture, molecular biology, biochemistry, or related fields are intended to be within the scope of the following claims.

What is claimed is:

1. A kit comprising:
a product dish operable to support an insert tray, the insert tray containing a polycarbonate membrane and an allogeneic cellularized scaffold product loosely adhered to the polycarbonate membrane.

2. The kit of claim 1, further comprising a hold solution and a hold dish.

3. The kit of claim 2, wherein the hold solution is packaged in a bottle contained within a laminated, foil pouch.

4. The kit of claim 2, wherein the hold dish comprises identical top and bottom portions.

5. The kit of claim 2, wherein the hold dish is packaged in a clear pouch.

6. The kit of claim 1, wherein the product dish is packaged in a foil pouch.

7. The kit of claim 1, wherein the polycarbonate membrane forms the bottom surface of the insert tray.

8. The kit of claim 1, wherein the insert tray further comprises polystyrene.

9. The kit of claim 1, wherein the allogeneic cellularized scaffold product comprises NIKS cells.

10. The kit of claim 9, wherein the allogeneic cellularized scaffold further comprises dermal fibroblasts.

11. The kit of claim 1, wherein the hold dish comprises a reservoir formed by a peripheral ledge on a bottom surface of the hold dish.

12. The kit of claim 12, wherein the reservoir is operable to contain the hold solution.

13. The kit of claim 13, wherein the insert tray is configured to rest on the peripheral ledge of the hold dish.

14. The kit of claim 1, wherein the product dish comprises a reservoir formed by a perimeter ledge on a bottom surface of the hold dish.
